(19) United States
(12) Patent Application Publication
(10) Pub. No.: US 2004/0038256 A1
(43) Pub. Date: Feb. 26, 2004

(54) Methods for identifying nucleotides at defined positions in target nucleic acids using fluorescence polarization

(75) Inventors: Jeffrey Van Ness, Claremont, CA (US); David J Galas, Claremont, CA (US); Lori K Garrison, Claremont, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: KECK GRADUATE INSTITUTE, Claremont, CA (US)

(21) Appl. No.: 10/398,101

(22) PCT Filed: Oct. 1, 2001
(86) PCT No.: PCT/US01/30743

(51) Int. Cl. C12Q 1/68

(52) U.S. Cl. 435/6

(57) ABSTRACT

The invention provides a method for identifying a nucleotide at a defined position in a target nucleic acid using restriction endonucleases and fluorescence polarization. The invention further provides compounds, compositions, and kits related to the method.

### BACKGROUND OF THE INVENTION

1. Field of the Invention

This invention relates to the field of molecular biology, more particularly to methods and compositions involving nucleic acids, and still more particularly to methods and compositions for identifying a particular nucleotide in a target nucleic acid.

2. Description of the Related Art

The chromosomal mapping and nucleic acid sequencing of each of the 80,000 to 100,000 human genes, achieved through the Human Genome Project, provides an opportunity for a comprehensive approach to the identification of nucleotide loci responsible for genetic diseases. Many of the 150-200 common genetic diseases and ˜600-800 of the rarer genetic diseases are associated with one or more defective genes. Of these, more than 200 human diseases are known to be caused by a defect in a single gene, often resulting in a change of a single amino acid residue. (Olsen, “Biotechnology: An Industry Comes of Age” (National Academic Press, 1986)).

Mutations occurring in somatic cells may induce disease if the mutations affect genes involved in cellular division control, resulting in, for example, tumor formation. In the germline, loss-of-function mutations in many genes can give rise to a detectable phenotype in humans. The number of cell generations in the germline, from one gamete to a gamete in an offspring, may be around 20-fold greater in the male germline than in the female. In the female, an egg is formed after a second meiotic division and lasts for 40 years. Therefore the incidence of different types of germline mutations and chromosomal aberrations depends on the parent of origin.

A majority of mutations, germline or somatic, are of little consequence to the organism since most of the genome appears to lack coding function (about 94%). Even within exon regions, there is some tolerance to mutations both due to the degeneracy of the genetic code and because the amino acid substitutions may have only a slight influence on a protein's function. (See, e.g., Strong et al., New England Journal of Medicine 325:1597 (1991)). With the development of increasingly efficient methods to detect mutations in large DNA segments, the need to predict the functional consequences (e.g., the clinical phenotype) of a mutation become more important.

While point mutations predominate among mutations in the human genome, individual genes may exhibit peculiar patterns of mutations and, accordingly, pose different diagnostic problems. In approximately 60% of cases of Duchenne muscular dystrophy, the mutation involves a deletion of a large segment of the gigantic dystrophine gene. The elucidated mutation causing the fragile X syndrome is characterized by an increased copy number of a particular repeated sequence (CCG)ₙ. Hereditarily unstable DNA of this type may prove to be a more general phenomenon in human disease than is generally recognized.

Molecular genetic techniques have not been employed to a significant extent in the diagnosis of chromosomal aberrations in genetic and malignant disease; cytogenetics remains the preferred technique to investigate these important genetic mechanisms. In an individual with one mutated copy of a tumor suppressor gene, the remaining normal allele may be replaced by a second copy of the mutant allele in one cell per 10³-10⁴. Mechanisms causing this replacement include chromosomal nondisjunction, mitotic recombination, and gene conversion. In contrast, independent mutations destroying the function of the remaining gene copy, are estimated to occur in one cell out of 10⁶.

Sensitive mutation detection techniques offer extraordinary possibilities for mutation screening. For example, analyses may be performed even before the implantation of a fertilized egg. (Holding et al., Lancet 3:532 (1989)). Increasingly efficient genetic tests may also permit screening for oncogenic mutations in cells exfoliated from the respiratory tract or the bladder in connection with health checkups. (Sidransky et al., Science 252:706, 1991). Alternatively, when an unknown gene causes a genetic disease, methods to monitor DNA sequence variants are useful to study the inheritance of disease through genetic linkage analysis. Notwithstanding these unique applications for the detection of mutations in individual genes, the existing methodology for achieving such applications continues to pose technological and economic challenges. While several different approaches have been pursued, none are sufficiently efficient and cost effective for wide scale application.

Conventional methods for detecting mutations at defined nucleotide loci involve time-consuming linkage analyses in families using limited sets of genetic markers that are difficult to “readout.” Such methods include, e.g., DNA marker haplotyping (that identifies chromosomes with an affected gene) as well as methods for detecting major rearrangements such as large deletions, duplications, translocations and single base pair mutations. These methods include scanning, screening and fluorescence resonance energy transfer (FRET)-based techniques. (See, Cotton, “Mutation Detection” (Oxford University Press, 1997)).

Highly sensitive assays that detect low abundance mutations rely on PCR to amplify the target sequence. Non-selective PCR strategies, however, amplify both mutant and wild-type alleles with approximately equal efficiency. Accordingly, low abundance mutant alleles are represented in only a small fraction of the final product. Thus, if the mutant sequence comprises <25% of the amplified product, it is unlikely that DNA sequencing approaches will be able to detect its presence. Although it is possible to quantify low abundance mutations by first separating the PCR products by cloning and subsequent probing of the clones with allele-specific oligonucleotides (ASOs), this approach is both labor intensive (requiring multiple lengthy procedures) and costly. (Saiki et al., Nature 324:163-166 (1986); Sidransky et al., Science 256:102-105 (1992); and Brennan et al., N. Engl. J Med. 332:429-435 (1995)).

In contrast to the above, allele-specific PCR methods can rapidly and preferentially amplify mutant alleles. For example, multiple mismatch primers have been used to detect H-ras mutations at a sensitivity of one mutant in 10⁵ wild-type alleles and sensitivity as high as one mutant in 10⁶ wild-type alleles have been reported. (Haliassos et al., Nucleic Acids Res. 17:8093-8099 (1989) and Chen et al., Anal. Biochem., 244:191-194 (1997)). These successes are, however, limited to allele-specific primers discriminating through 3′ purine.purine mismatches. For the more common transition mutations, the discriminating mismatch on the 3′ primer end (i.e., G:T or C:A mismatch) will be removed in a small fraction of products by polymerase error during extension from the opposite primer on wild-type DNA. Thereafter, these error products are efficiently amplified and generate false positive signals.

It has been suggested that one means to eliminate the polymerase error problem is to deplete wild-type DNA early in the amplification cycles. Several reports have explored selective removal of wild-type DNA by restriction endonuclease digestion in order to enrich for low abundance mutant sequences. These restriction fragment length polymorphism (RFLP) methods detect approximately one mutant in 10⁶ wild-type or better. One approach has employed digestion of genomic DNA followed by PCR amplification of the uncut fragments (RFLP-PCR) to detect very low level mutations within restriction sites in the H-ras and p53 genes. (Sandy et al., Proc. Natl Acad Sci. USA 89:890-894 (1992) and Pourzand et al., Mutat. Res. 288:113-121 (1993)). Similar results have been obtained by digestion following PCR and subsequent amplification of the un-cleaved DNA now enriched for mutant alleles (PCR-RFLP). (Kumar et al., Oncogene 3:647-651 (1988); Kumar et al., Oncogene Res., 4:235-241 (1989) and Jacobson et al., Oncogene 9:553-563 (1994)).

Although sensitive and rapid, RFLP detection methods are limited by the requirement that the location of the mutations must coincide with restriction endonuclease recognition sequences. To circumvent this limitation, primers that introduce a restriction site (part of the recognition sequence is in the template DNA) have been employed in “primer-mediated RFLP.” (Jacobson et al., PCR Methods Applicat. 1:299 (1992); Chen et al., Anal. Biochem. 195:51-56 (1991); Di Giuseppe et al., Am. J. Pathol. 144:889-895 (1994); Kahn et al., Oncogene 6:1079-1083 (1991); Levi et al., Cancer Res. 51:3497-3502 (1991) and Mitsudomi et al., Oncogene 6:1353-1362 (1991)). Subsequent investigators have demonstrated, however, that errors are produced at the very next base by polymerase extension from primers having 3′ natural base mismatches. (Hattori et al., Biochem. Biophys. Res. Commun. 202:757-763 (1994); O'Dell et al., Genome Res. 6:558-568 (1996) and Hodanova et al., J. Inherit. Metab. Dis., 20:611-612 (1997)). Such templates fail to cleave during restriction digestion and amplify as false positives that are indistinguishable from true positive products extended from mutant templates.

Use of nucleotide analogs may reduce errors resulting from polymerase extension and improve base conversion fidelity. Nucleotide analogs that are designed to base pair with more than one of the four natural bases are termed “convertides”. Base incorporation opposite different convertides has been tested. (Hoops et al., Nucleic Acids Res. 25:4866-4871 (1997)). For each analog, PCR products were generated using Taq DNA polymerase and primers containing an internal nucleotide analog. The products generated showed a characteristic distribution of the four bases incorporated opposite the analogs.

Due, in part, to the shortcomings in the existing methodology for detecting genetic mutations, there exists an unmet need for rapid and sensitive methods for detecting mutations and parallel measurement of genetic variations. The present invention fulfills this and other related needs by providing methods for parallel measurement of genetic variations that, inter alia, display increased speed, convenience and specificity. As disclosed in detail herein below, methods according to the present invention are based on the incorporation of unique restriction endonuclease restriction sites flanking and/or encompassing genetic variation loci. These methods exploit the high degree of specificity afforded by restriction endonucleases and employ readily available detection techniques.

### SUMMARY OF THE INVENTION

The present invention provides various compounds, compositions and kits useful for, and method of, identifying genetic variations at, defined positions in target nucleic acids.

In one aspect, the present invention provides a method for identifying a nucleotide at a defined position in a single-stranded target nucleic acid, comprising

(a) forming a mixture of a first oligonucleotide primer (ODNP), a second ODNP, and the target nucleic acid, wherein

the first ODNP comprises a nucleotide sequence that is complementary to a nucleotide sequence of the target nucleic acid at a location 3′ to the defined position,

the second ODNP comprises a nucleotide sequence that is complementary to a nucleotide sequence of the complement of the target nucleic acid at a location 3′ to the complementary nucleotide of the nucleotide at the defined position, and

the first and second ODNPs further comprise a first constant recognition sequence (CRS) of a first strand and a second CRS of a second strand of an interrupted restriction endonuclease recognition sequence (IRERS), respectively, but not a complete IRERS, the complete IRERS being a double-stranded nucleic acid having the first and the second strands and comprising the first and the second constant recognition sequences (CRS) linked by a variable recognition sequence (VRS);

(b) extending the first and second ODNPs to form a fragment having the complete IRERS wherein the nucleotide to be identified is within the VRS;

(c) cleaving the fragment with a restriction endonuclease that recognizes the complete IRERS, and thereby producing a 5′-overhang consisting of either the nucleotide to be identified or the complement thereof;

(d) filing in the 3′ recessed terminus corresponding to the 5′ overhang with a fluorescence labeled nucleoside triphosphate; and

(e) detecting the incorporated fluorescence labeled nucleotide with fluorescence polarization.

In certain embodiments, the defined position is polymorphic. In some embodiments, a mutation at the defined position is associated with a disease, such as a human genetic disease that includes, but is not limited to, bladder carcinoma, colorectal tumors, sickle-cell anemia, thalassemias, a1-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease. In other embodiments, a mutation at the defined position is associated with drug resistance of a pathogenic microorganism.

In some embodiment, the single-stranded target nucleic acid is one strand of a denatured double-stranded nucleic acid. The double-stranded nucleic acid may be genomic nucleic acid, cDNA or synthetic nucleic acid. In certain embodiment, the single-stranded target nucleic acid is derived from the genome of a pathogenic virus. In another embodiment, the single-stranded target nucleic acid is derived from the genome or episome of a pathogenic bacterium. In yet another embodiment, the target nucleic acid is synthetic nucleic acid.

In some embodiments, either the nucleotide sequence of the first ODNP complementary to the target nucleic acid, or the nucleotide sequence of the second ODNP complementary to the complement of the target nucleic acid is at least 6, 8, 10, 12, 14 or 16 nucleotides in length.

In certain embodiments, either the first ODNP, or the second ODNP, or both ODNPs are 8-100 nucleotides in length, more preferably 15-85 nucleotides in length. The first ODNP may further comprise one or more nucleotides complementary to the target nucleic acid at the 3′ terminus of the first CRS. Similarly, the second ODNP may further comprise one or more nucleotides complementary to the target nucleic acid at the 3′ terminus of the second CRS. In some preferred embodiments, the 5′ terminus of either the first ODNP or the second ODNP is linked to a biotin molecule.

In certain embodiments, step (b) is carried out by performing a polymerase chain reaction. In some embodiments, step (d) is carried out with a RNA polymerase. Alternatively, step (d) may be carried out with either deoxynucleoside triphosphate or dideoxynucleoside triphosphate using a DNA polymerase.

In certain embodiments, the fluorescence labeled dideoxynucleoside triphosphate is any one selected from the group consisting of FAM-ddA, FAM-ddU, FAM-ddC, FAM-ddG, BODIPY-fluorescein-ddA (BFL-ddA), tetramethylrhodamine-ddC (TMR-ddC), b-caboxy-x-prhodamine-ddG (ROX-ddG), and BODIPY-Texas Red-ddU (BTR-ddU).

In some embodiments, the present method further comprises separating products of step (d) before the detection of step (e).

In certain embodiments, the IRERS is recognizable by a restriction endonuclease selected from the group consisting of PflF I, EcoN I, Fnu4H I, ScrF I, and Tth111 I.

Another aspect of the present invention provides an oligonucleotide primer, comprising:

(a) a first CRS of a first strand of an IRERS, but not the first strand of a complete IRERS, wherein the complete IRERS is a double-stranded oligonucleotide having the first strand and a second strand, the complete IRERS comprises the first CRS and a second CRS linked by a VRS having a number n of variable nucleotides, and the digestion at the IRERS produces a 5′ overhang consisting of a single nucleotide; and

(b) at a location 5′ to the 5′ terminus of the first CRS, an oligonucleotide sequence complementary to a nucleotide sequence of a single-stranded target nucleic acid at a location 3′ to a defined position, wherein when the oligonucleotide sequence anneals to the target nucleic acid, the distance between the nucleotide in the target corresponding to the 3′ terminal nucleotide of the primer and the defined position is within the range 0 to (n−1)/2.

In certain embodiments, oligonucleotide sequence (b) is at least 6, 8, 10, 12, 14, or 16 nucleotides in length. In some embodiments, the primer is 8-200 nucleotides in length. In other preferred embodiments, the primers are 15-85 or 18-32 nucleotide in length. The primer may further comprise one or more nucleotides complementary to the target nucleic acid at the 3′ terminus of the first CRS. In certain preferred embodiments, the 5′ terminus of the primer is linked to a biotin molecule. The IRERS may be recognizable by EcoN I.

Preferably, the defined position in the target nucleic acid is polymorphic. In some embodiments, a mutation at the defined position in the target nucleic acid is associated with a disease. The target nucleic acid may one strand of a denatured double-stranded nucleic acid, including genomic nucleic acid and cDNA.

Another aspect of the present invention provides an oligonucleotide primer pair for producing a portion of a single-stranded target nucleic acid containing a nucleotide to be identified at a defined position, comprising first and second ODNPs wherein

the first ODNP comprises a nucleotide sequence complementary to a nucleotide sequence of the target nucleic acid at a location 3′ to the defined position;

the second ODNP comprises a nucleotide sequence complementary to a nucleotide sequence of the complement of the target nucleic acid at a location 3′ to the complementary nucleotide of the nucleotide to be identified;

the first and second ODNPs further comprise a first constant recognition sequence (CRS) of a first strand and a second CRS of a second strand of an interrupted restriction endonuclease recognition sequence (IRERS), respectively, but not a complete IRERS, the complete IRERS being a double-stranded nucleic acid having the first and the second strands and comprising the first and the second constant recognition sequences (CRS) linked by a variable recognition sequence (VRS); and

a fragment resulting from an amplification of the first and second ODNPs comprises a complete IRERS, and digestion at the IRERS produces a 5′ overhang consisting of either the nucleotide to be identified or the complement thereof.

In some embodiments, either the nucleotide sequence complementary to the target nucleic acid of the first ODNP, or the nucleotide sequence complementary to the complement of the target nucleic acid of the second ODNP, or both, are at least 6, 8, 10, 12, 14, or 16 nucleotides in length. Preferably, the IRERS is recognizable by EcoN I.

In certain embodiments, either the first ODNP, or the second ODNP, or both ODNPs are 8-100 nucleotides in length, preferably 15-85 nucleotides in length. Preferably, the first ODNP may further comprise one or more nucleotides complementary to the target nucleic acid at the 3′ terminus of the first CRS. Likewise, the second ODNP may further comprise one or more nucleotides complementary to the target nucleic acid at the 3′ terminus of the second CRS. Preferably, the 5′ terminus of either the first or the second ODNP is linked to a biotin molecule.

The defined position in the target nucleic acid may be polymorphic or associated with a disease. The target nucleic acid may be one strand of a denatured double-stranded nucleic acid, such as genomic nucleic acid and cDNA.

The present invention provides a composition comprising the primer and the target nucleic acid as described above. It further provides a kit comprising the above primer pair. The kit may further comprise a restriction endonuclease that recognizes the IRERS a portion of which constitutes partial sequences of the primer pair. The kit may also further comprise instruction of use thereof.

In another aspect, the present invention provides a set of two ODNP pairs, comprising first and second ODNP pairs each comprising first and second ODNPs wherein:

(a) the first ODNP in the first ODNP pair comprises

an oligonucleotide sequence complementary to a nucleotide sequence of a single-stranded target nucleic acid at a location 3′ to a defined position in the target nucleic acid, and

a first CRS of a first strand of an IRERS, but not the first strand of a complete IRERS, the complete IRERS being a double-stranded nucleic acid having first and second strands and comprising the first CRS and a second CRS linked by a VRS;

(b) the second ODNP in the first ODNP pair comprises

an oligonucleotide sequence complementary to a nucleotide sequence of the target nucleic acid at a location 5′ to the defined position, and

a second CRS of the first strand of the IRERS, but not the first strand of the complete IRERS;

(c) the first ODNP in the second ODNP pair comprises

an oligonucleotide sequence complementary to a nucleotide sequence of the complement of the target nucleic acid at a location 5′ to the position in the complement corresponding to the defined position in the target nucleic acid, and

a first CRS of the second strand of the IRERS, but not the second strand of the complete IRERS; and

(d) the second ODNP in the second ONDP pair comprises

an oligonucleotide sequence complementary to a nucleotide sequence of the complement of the target nucleic acid at a location 3′ to the position in the complement corresponding to the defined position in the target nucleic acid, and

a second CRS of the second strand of the IRERS, but not the second strand of the complete IRERS; and

(e) a fragment resulting from an extension and ligation of the first and second ODNPs in each ODNP pair comprises the complete IERS, wherein digestion at the IRERS produces a 5′ overhang consisting of either the nucleotide at the defined position or the complement thereof.

In yet another aspect, the present invention provides a method comprising:

(a) providing a double-stranded nucleic acid molecule comprising an interrupted restriction endonuclease recognition sequence (IRERS), wherein the IRERS comprises a first constant recognition sequence (CRS) and a second CRS linked by a variable recognition sequence (VRS);

(b) cleaving the nucleic acid molecule with a restriction endonuclease that recognizes the IRERS, and thereby producing a fragment with a 5′ overhang consisting of either a nucleotide to be identified at a defined position in the nucleic acid molecule or the complementary nucleotide thereof;

(c) filing in the 3′ recessed terminus corresponding to the 5′ overhang with a fluorescence labeled nucleoside triphosphate; and

(d) detecting the incorporated fluorescence labeled nucleotide with fluorescence polarization.

In some embodiment, step (a) comprises:

(i) forming a mixture of the primer pair set of the present invention as described above and the target nucleic acid;

(ii) extending the first and second ODNPs of the first and second ODNP pairs;

(iii) ligating the extended products of step (ii); and

(iv) amplifying the fragments of step (iii).

In another embodiment, step (a) comprises:

(i) forming a mixture of the primer pair of the present invention as described above and the target nucleic acid; and

(ii) extending the first and the second ODNPs.

In yet another embodiment, step (a) comprises:

(i) forming a mixture of a first ODNP, a second ODNP and a single-stranded target, wherein

the first ODNP comprises

an oligonucleotide sequence complementary to a nucleotide sequence of the target nucleic acid at a location 3′ to a defined position in the target nucleic acid, and

a first CRS of a first strand of an IRERS, but not the first strand of a complete IRERS; the complete IRERS being a double-stranded nucleic acid having first and second strands and comprising the first CRS and a second CRS linked by a VRS,

the second ODNP comprises

an oligonucleotide sequence complementary to a nucleotide sequence of the target nucleic acid at a location 5′ to the defined position, and

a second CRS of the first strand of the IRERS, but not the first strand of the complete IRERS;

(ii) extending the first and second ODNPs;

(iii) ligating the extended products of step (ii);

(iv) denaturing the ligation product of step (iii); and

(v) annealing the denatured ligation product of step (iv) that contains said first and second ODNPs with an oligonucleotide that has a universe nucleotide at the position corresponding to the defined position in the double stranded nucleic acid molecule, wherein the resulting double-stranded nucleic acid molecule comprises an complete IRERS.

In certain embodiments, the defined position is polymorphic. In some embodiments, a mutation at the defined position is associated with a disease, such as a human genetic disease that includes, but is not limited to, bladder carcinoma, colorectal tumors, sickle-cell anemia, thalassemias, a1-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease. In other embodiments, a mutation at the defined position is associated with drug resistance of a pathogenic microorganism.

In some embodiments, step (d) is carried out with a RNA polymerase. Alternatively, step (d) may be carried out with either deoxynucleoside triphosphate or dideoxynucleoside triphosphate using a DNA polymerase.

In certain embodiments, the fluorescence labeled dideoxynucleoside triphosphate is any one selected from the group consisting of FAM-ddA, FAM-ddU, FAM-ddC, FAM-ddG, BODIPY-fluorescein-ddA (BFL-ddA), tetramethylrhodamine-ddC (TMR-ddC), b-caboxy-x-prhodamine-ddG (ROX-ddG), and BODIPY-Texas Red-ddU (BTR-ddU).

In some embodiments, the present method further comprises separating products of step (d) before the detection of step (e).

In certain embodiments, the IRERS is recognizable by a restriction endonuclease selected from the group consisting of PflF I, EcoN I, Fnu4H I, ScrF I, and Tth111 I.

In another aspect, the present invention provides a method comprising the steps:

(a) combining a first ODNP, a second ODNP, and a single stranded target nucleic acid under primer extension conditions, wherein

the first ODNP comprises

an oligonucleotide sequence complementary to a nucleotide sequence of the target nucleic acid at a location 3′ to a defined position in the a target nucleic acid, and

a first CRS of a first strand of an IRERS, but not the first strand of a complete IRERS, the complete IRERS being a double-stranded nucleic acid having first and second strands and comprising the first CRS and a second CRS linked by a VRS,

the second ODNP comprises

an oligonucleotide sequence complementary to a nucleotide sequence of the target nucleic acid at a location 5′ to the defined position, and

a second CRS of the first strand of the IRERS, but not the first strand of the complete IRERS;

(b) performing at least three rounds of primer extension to provide a primer extension product;

(c) cleaving the primer extension product with a restriction endonuclease that recognizes an IRERS and thereby producing a 5′ overhang consisting of either the nucleotide at the defined position or the complement thereof;

(d) filling in the 3′ recessed terminus corresponding to the 5′ overhang with a fluorescence labeled nucleoside triphosphate; and

(e) detecting the incorporated fluorescence labeled nucleotide with fluorescence polarization.

In some embodiment, the defined position is associated with a disease. In certain embodiment, the single-stranded target nucleic acid is one strand of a denatured double-stranded nucleic acid. The double-stranded nucleic acid may be genomic nucleic acid, cDNA or synthetic nucleic acid.

In some embodiments, either the nucleotide sequence of the first ODNP complementary to the target nucleic acid, or the nucleotide sequence of the second ODNP complementary to the complement of the target nucleic acid is at least 6, 8, 10, 12, 14 or 16 nucleotides in length.

The first ODNP may further comprise one or more nucleotides complementary to the target nucleic acid at the 3′ terminus of the first CRS. Similarly, the second ODNP may further comprise one or more nucleotides complementary to the target nucleic acid at the 3′ terminus of the second CRS. Preferably, the 5′ terminus of either the first ODNP or the second ODNP is linked to a biotin molecule.

In certain embodiments, step (b) is carried out by performing a polymerase chain reaction. In some embodiments, step (d) is carried out with a RNA polymerase. Alternatively, step (d) may be carried out with either deoxynucleoside triphosphate or dideoxynucleoside triphosphate using a DNA polymerase.

In certain embodiments, the fluorescence labeled dideoxynucleoside triphosphate is any one selected from the group consisting of FAM-ddA, FAM-ddU, FAM-ddC, FAM-ddG, BODIPY-fluorescein-ddA (BFL-ddA), tetramethylrhodamine-ddC (TMR-ddC), b-caboxy-x-prhodamine-ddG (ROX-ddG), and BODIPY-Texas Red-ddU (BTR-ddU).

In some embodiments, the present method further comprises separating products of step (d) before the detection of step (e).

In certain embodiments, the IRERS is recognizable by a restriction endonuclease selected from the group consisting of PflF I, EcoN I, Fnu4H I, ScrF I, and Tth111 I.

### BRIEF DESCRIPTION OF THE DRAWINGS

FIGS. 1A and 1B together are a diagram of major steps of the present method for identifying a nucleotide at a defined position in a target nucleic acid using two ODNPs and the restriction endonuclease recognition sequence for EcoN I.

FIG. 2 shows changes in fluorescence polarization for DNA samples genotyped with FP-EcoN I assay.

FIG. 3 is a schematic diagram of the major components of the ODNPs and a resulting amplicon of the present invention.

FIG. 4 is a schematic diagram of the major components of an interrupted restriction endonuclease recognition sequence. A₁A₂ . . . Aₘ is a specific nucleotide sequence consisting of m nucleotides, whereas A′₁A′₂ . . . A′ₘ is the complement sequence of A₁A₂ . . . Aₘ. The double-stranded fragment comprised of A₁A₂ . . . Aₘ and A′₁A′₂ . . . A′ₘ forms the first CRS (also referred to as “Region A”). N₁N₂ . . . Nₙ is a variable nucleotide sequence consisting of n nucleotides where any one of the nucleotide can contain any of the four bases (a, c, t, or g). N′₁N′₂ . . . N′ₙ is the complement of N₁N₂ . . . Nₙ and forms a VRS (also referred to “Region B” where the number n is equal to the number B) in combination of N₁N₂ . . . Nₙ. C₁C₂ . . . Cᵢ is a specific nucleotide sequence consisting of i nucleotides, whereas C′₁C′₂ . . . C′ᵢ is the complement of C₁C₂ . . . . Cᵢ. The double-stranded fragment comprised of C₁C₂ . . . . Cᵢ and C′₁C′₂ . . . C′ᵢ forms the second CRS (also referred to as “Region C”).

FIG. 5 is a schematic diagram of a set of two ODNP pairs.

FIG. 6 is a schematic diagram of major steps in the present method for identifying a nucleotide at a defined position in a target nucleic acid using a set of two ODNP pairs and the exemplary restriction endonuclease recognition sequence for EcoN I.

FIG. 7 is a schematic diagram of major steps of one embodiment of the present method for providing a double-stranded nucleic acid molecule containing an IRERS.

### DETAILED DESCRIPTION OF THE INVENTION

The present invention provides methods, compositions, and kits for determining sequence information at a defined genetic locus in a target nucleic acid. As described in more detail below, the invention provides for the design, preparation and use of oligonucleotide primers (ODNPs) that can be extended in a manner that incorporates information about the nucleotide of interest into the extension product. The resulting product, e.g., amplicon, can then be analyzed by fluorescence polarization, also described in more detail below, to determine the identity of the nucleotide of interest. This information is advantageously utilized in a variety of applications, as described herein, such as genetic analysis for hereditary diseases, tumor diagnosis, disease predisposition, forensics or paternity, crop cultivation and animal breeding, expression profiling of cell function and/or disease marker genes, and identification and/or characterization of infectious organisms that cause infectious diseases in plants or animals and/or that are related to food safety.

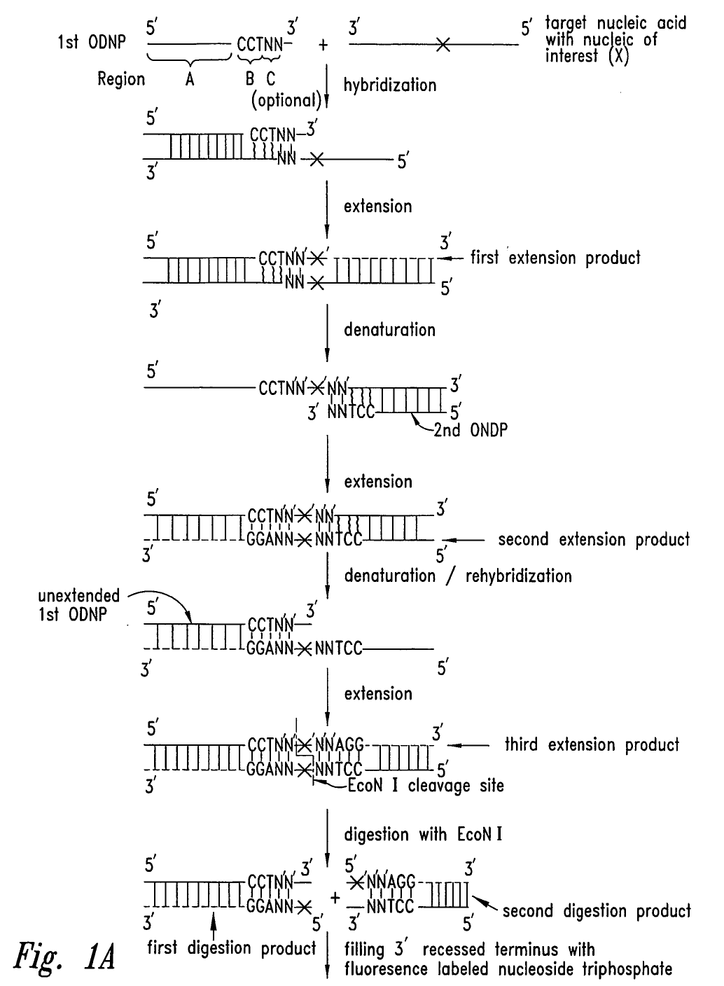

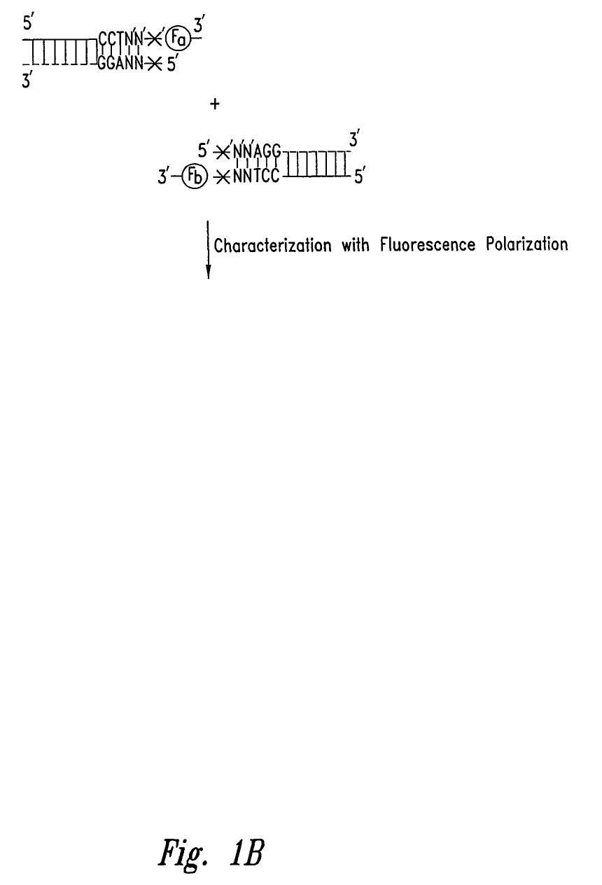

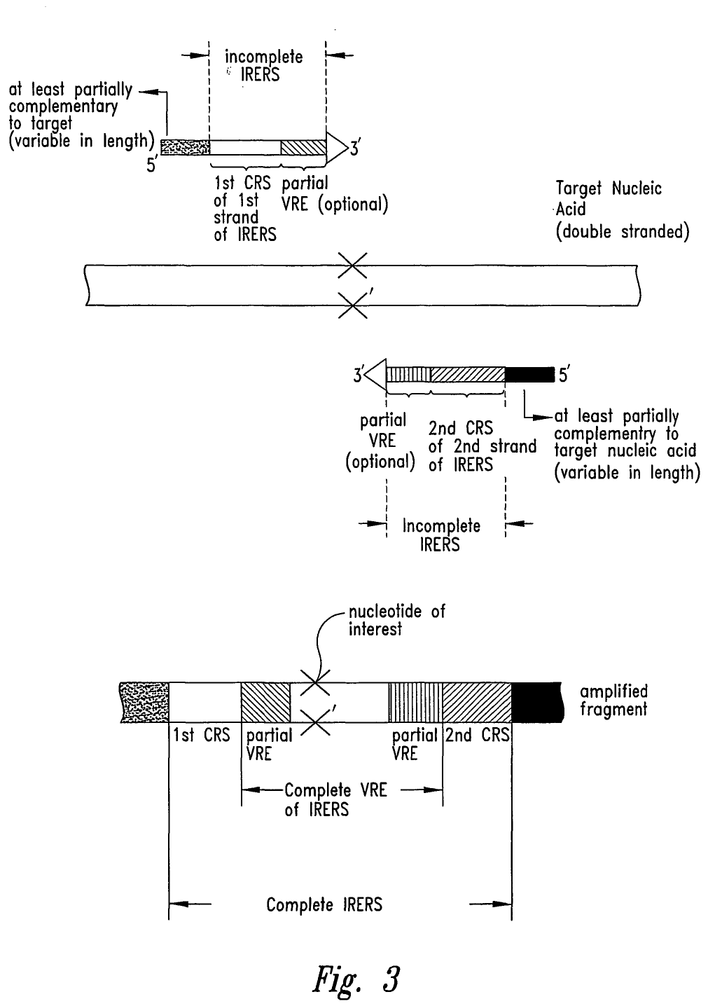

The ODNPs of the present invention each contain part of an interrupted restriction endonuclease recognition sequence (IRERS), defined in detail below. The interrupted segment of the restriction endonuclease recognition site (also referred to as “variable recognition sequence (VRS)”) may be one or more nucleotides in length and the sequence is variable (each position can contain any of the four bases (a, c, t, or g)). When extended and incorporated into an amplified fragment, the two primers together, in combination with the segment of target nucleic acid between them (i.e., VRS) form a single and complete IRERS. In addition, the complete IRERS of the present invention is selected so that digestion of a nucleic acid fragment containing such an IRERS by a restriction endonuclease that recognizes the IRERS produces digestion products with 5′ overhang. The primers are designed such that the nucleotide of interest in a target nucleic acid is located in the amplicon within both the variable segment of the restriction endonuclease recognition site and 5′ overhangs produced by digestion at the IRERS. The amplicon can then be digested to generate small fragments of nucleic acid that can be analyzed to determine the nucleotide of interest with great accuracy and sensitivity. The oligonucleotide primers of the present invention are shown schematically in FIG. 3. In FIG. 1, a diagram of the present invention is shown using the exemplary restriction endonuclease recognition sequence for EcoN I. One skilled in the art will appreciate that any interrupted restriction endonuclease recognition sequence may be used at which digestion produces a 5′ overhang (preferably a 5′ overhang consisting of a single nucleotide) may be used.

In various aspects, the present invention provides assays for determining the identity of a base at a predetermined location in a target nucleic acid molecule. In additional aspects, provided herein are compounds, compositions, and kits that are useful in performing such assays.

A. Conventions

Prior to providing a more detailed description of the present invention, it may be helpful to an understanding thereof to define a convention as used herein, as follows. The terms “3′” and “5′” are used herein to assist in describing the location of a particular site within a single strand of nucleic acid. When a location in a nucleic acid is “3′ to” or “3′ of” a nucleotide of interest, this means that it is between the nucleotide of interest and the 3 hydroxyl of that strand of nucleic acid. Likewise, when a location in a nucleic acid is “5′to” or “5′of” a nucleotide of interest, this means that the location is between the nucleotide of interest and the 5′ phosphate of that strand of nucleic acid.

As used herein, a nucleotide in the complement of a single-stranded target nucleic acid at a position “corresponding to” a defined position in the target refers to a nucleotide complementary to the nucleotide at the defined position in the target.

Also, as used herein, the word “a” refers to one or more of the indicated items unless the context clearly indicates otherwise. For instance, “a” polymerase refers to one or more polymerases.

B. Methodology of the Present Invention

According to the present invention, the identity of a nucleotide of interest in a target nucleic acid molecule is determined by combining the target with two primers, where one molecule of the first primer hybridizes to, and extends from, a location 3′ of the nucleotide of interest in the target, so as to incorporate the complement of the nucleotide of interest in a first extension product. One molecule of the second primer then hybridizes to, and extends based on, the first extension product, at a location 3′ of the complement of the nucleotide of interest, so as to incorporate the nucleotide of interest in a second extension product. Another molecule of the first primer then hybridizes to, and extends from, a location 3′ of the nucleotide of interest in the second extension product, so as to form, in combination with the second extension product, a nucleic acid fragment. The first and second primers are designed to each incorporate a portion of the recognition sequence of an IRERS, the IRERS being selected so that digestion at the IRERS produces fragments with 5′ overhangs. In addition, the primers are also designed such that the nucleotide of interest in the target is located within both the VRS and a 5′ overhang produced by digestion at the IRERS. The 3′ recessed termini corresponding to 5′ overhangs produced by digestion at the IRERS are then filled in with one or more fluorescence labeled nucleoside triphosphates. The incorporated nucleotide(s) can be detected using the technique of fluorescence polarization.

1. Target Nucleic Acid Molecules

Methods, kits and compositions of the present invention typically involve or include a target nucleic acid molecule. The target nucleic acid of the present invention is any nucleic acid molecule about which nucleotide information is desired, and which can serve as a template for a primer extension reaction, e.g., can base pair with a primer. The target nucleic acid may also be referred to as the template nucleic acid.

The term “nucleic acid” refers generally to any molecule, preferably a polymeric molecule, incorporating units of ribonucleic acid or an analog thereof. The template nucleic acid can be either single-stranded or double-stranded. In one aspect, the template nucleic acid is DNA. In another aspect, the template is RNA. Suitable nucleic acid molecules are DNA, including genomic DNA, ribosomal DNA and cDNA. Other suitable nucleic acid molecules are RNA, including mRNA, rRNA and tRNA. The nucleic acid molecule may be naturally occurring, as in genomic DNA, or it may be synthetic, i.e., prepared based up human action or intervention, or may be a combination of the two.

A naturally occurring nucleic acid is obtained from a biological sample. The biological sample can be any sample that contains biological material (e.g., cells, tissues) from any organisms, including but not limited to, animals, higher plants, fungi, bacteria, and viruses. Mammalian tissue (for example blood, plasma/serum, hair, skin, lymph node, spleen, liver, etc) and/or mammalian cells or cell lines are a preferred type of biological sample. The biological sample may comprise one or more different human tissues and/or cells. Mammalian and/or human tissues and/or cells may further comprise one or more tumor tissues and/or cells. Another type of preferred biological sample is or contains a virus, such as pathogenic viruses (e.g., hepatitis A, B, or C), herpes virus (e.g., VZV, HSV-1, HAV-6, HSV-II, CMV, and Epstein Barr virus), adenovirus, influenza virus, echovirus, rhinovirus, cornovirus, respiratory syncytical virus, mumps virus, measles virus, rubella virus, parvovirus, poliovirus, rabies virus, and flaviviruses. Another preferred biological sample contains genomic or episomic nucleic acids of pathogenic bacteria, particularly regions conferring drug resistance or useful for phylogenic characterization of the host (e.g., 16S rRNA). Such bacteria include, but are not limited to, chlamydia, rickettsial bacteria, mycobacteria, staphylococci, treptocci, pneumonococci, meningococci, conococci, klebsiella, proteus, serratia, pseudomonas, bacilli, cholera, tetanus, anthrax, plague, and Lymes disease bacteria.

Methodology for isolating populations of nucleic acids from biological samples is well known and readily available to those skilled in the art of the present invention. Exemplary techniques are described, for example, in the following laboratory research manuals: Sambrook et al., “Molecular Cloning” (Cold Spring Harbor Press, 3rd Edition, 2001) and Ausubel et al., “Short Protocols in Molecular Biology” (1999) (incorporated herein by reference in their entirety). Nucleic acid isolation kits are also commercially available from numerous companies which simplify and accelerate the isolation process.

A synthetic nucleic acid is produced by human intervention. At this time, many companies are in the business of making and selling synthetic nucleic acids that may be useful as the template nucleic acid molecule in the present invention. See, e.g., Applied Bio Products Bionexus (www.bionexus.net); Blue Heron Biotechnology (Bothell, Wash.; www.blueheron.com); Commonwealth Biotechnologies, Inc. (Richmond, Va.; www.cbi-biotech.com); Gemini Biotech (Alachua, Fla.; www.geminibio.com); INTERACTIVA Biotechnologie GmbH (Ulm, Germany; www.interactiva.de); Microsynth (Balgach, Switzerland; www.microsynth.ch); Midland Certified Reagent Company (Midland, Tex.; www.mcrc.com); Oligos Etc. (Wilsonville, Oreg.; www.oligosetc.com); Operon Technologies, Inc. (Alameda, Calif.; www.operon.com); Scandanavian Gene Synthesis AB (Koping, Sweden; www.sgs.dna); Sigma-Genosys (The Woodlands, Tex.; www.genosys.com); Synthetic Genetics (San Diego, Calif.; www.syntheticgenetics.com, which was recently purchased by Epoch Biosciences, Inc. (Bothell, Wash.; www.epochbio.com); and many others.

The synthetic nucleic acid template may be prepared using an amplification reaction. The amplification reaction may be, for example, the well-known polymerase chain reaction (PCR).

The synthetic nucleic acid template may be prepared using recombinant DNA means through production in one or more prokaryotic or eukaryotic organism such as, e.g., E. coli, yeast, Drosophila or mammalian tissue culture cell line.

The nucleic acid molecule may, and typically will, contain one or more of the ‘natural’ nucleotide bases, i.e., adenine (A), guanine (G), cytosine (C), thymine (T) and, in the case of an RNA, uracil (U). In addition, and particularly when the nucleic acid is a synthetic molecule, the target nucleic acid may include “unnatural” nucleotides. Unnatural nucleotides are chemical moieties that can be substituted for one or more natural nucleotides in a nucleotide chain without causing the nucleic acid to lose its ability to serve as a template for a primer extension reaction. The substitution may include either sugar and/or phosphate substitutions, in addition to base substitutions.

Such moieties are very well known in the art, and are known by a large number of names including, for example, abasic nucleotides, which do not contain a commonly recognized nucleotide base, such as adenine, guanine, cytosine, uracil or thymine (see, e.g., Takeshita et al. “Oligonucleotides containing synthetic abasic sites” The Journal of Biological Chemistry, vol. 262, pp. 10171-10179 1987; Iyer et al. “Abasic oligodeoxyribonucleoside phosphorothioates: synthesis and evaluation as anti-HIV-1 agents” Nucleic acids Research, vol. 18, pp. 2855-2859 1990; and U.S. Pat. No. 6,117,657); base or nucleotide analogs (see, e.g., Ma et al., “Design and Synthesis of RNA Miniduplexes via a Synthetic Linker Approach. 2. Generation of Covalently Closed, Double-Stranded Cyclic HIV-1 TAR RNA Analogs with High Tat-Binding Affinity,” Nucleic Acids Research 21:2585 (1993). Other exemplary unnatural bases include universal mismatch base analogs, (such as the abasic 3-nitropyrrole); convertides (see, e.g., Hoops et al., Nucleic Acids Res. 25:4866-4871 (1997)); modified nucleotides (see, e.g., Millican et al., “Synthesis and biophysical studies of short oligodeoxynucleotides with novel modifications: A possible approach to the problem of mixed base oligodeoxynucleotide synthesis,” Nucleic Acids Research 12:7435-7453 (1984); nucleotide mimetics; nucleic acid related compounds; spacers (see, e.g., Nielsen et al. Science, 254:1497-1500 (1991); and specificity spacers (see, e.g., PCT International Publication No. WO 98/13527).

Additional examples of non-natural nucleotides are set forth in: Jaschke et al., Tetrahedron Lett., 34:301 (1993); Seela and Kaiser, Nucleic Acids Research 15:3113 (1990) and Nucleic Acids Research 18:6353 (1990); Usman et al., PCT International Patent Application No. PCT/US 93/00833; Eckstein, PCT International Patent Application No. PCT/EP91/01811; Sproat et al., U.S. Pat. No. 5,334,711, and Buhr and Matteucci, PCT International Publication No. WO 91/06556; Augustyns, K. A. et al., Nucleic Acids Res., 1991, 19, 2587-2593); and U.S. Pat. Nos. 5,959,099 and 5,840,876.

When the template nucleic acid molecule, and/or the primer used in the present method, contains a non-natural nucleotide, then a base-pair mismatch will occur between the template and the primer. The term “base-pair mismatch” refers to all single and multiple nucleotide substitutions that perturb the hydrogen bonding between conventional base pairs, e.g., G:C, A:T, or A:U, by substitution of a nucleotide with a moiety that does not hybridize according to the standard Watson-Crick model to a corresponding nucleotide on the opposite strand of the oligonucleotide duplex. Such base-pair mismatches include, e.g., G:G, G:T, G:A, G:U, C:C, C:A, C:T, C:U, T:T, T:U, U:U and A:A. Also included within the definition of base-pair mismatches are single or multiple nucleotide deletions or insertions that perturb the normal hydrogen bonding of a perfectly base-paired duplex. In addition, base-pair mismatches arise when one or both of the nucleotides in a base pair has undergone a covalent modification (e.g., methylation of a base) that disrupts the normal hydrogen bonding between the bases. Base-pair mismatches also include non-covalent modifications such as, for example, those resulting from incorporation of intercalating agents such as ethidium bromide and the like that perturb hydrogen bonding by altering the helicity and/or base stacking of an oligonucleotide or polynucleotide duplex.

Among the nucleotide and, optionally, nucleotide analogs that form the target nucleic acid, there is one or more natural nucleotide(s) of unknown identity. The present invention provides kits, compositions and methods whereby the identity of the unknown nucleotide(s) becomes known. The nucleotide of unknown identity at the “nucleotide loci” (or the “defined position”) refers to a specific nucleotide having a precise location on a target nucleic acid.

The nucleotide to be identified in the target nucleic acid may be a mutation. The term “mutation” refers to an alteration in a wild-type nucleic acid sequence. Mutations may be in non-coding regions (introns or 5′ and 3′ flanking regions) or may be in regions encoding proteins (exons) of a target nucleic acid. Exemplary mutations in non-coding regions include regulatory mutations that alter the amount of gene product, localization of protein and/or timing of expression. Exemplary mutations in coding regions include nonsense mutations and missense mutations. A “nonsense mutation” is a single nucleotide change resulting in a triplet codon (where mutation occurs) being read as a “STOP” codon causing premature termination of peptide elongation, i.e., a truncated peptide. A “missense mutation” is a mutation that results in one amino acid being exchanged for a different amino acid in the gene's peptide expression product. Such a mutation may cause a change in the folding (3-dimensional structure) of the peptide and/or its proper association with one or more other peptides in a multimeric structure.

The nucleotide of interest, i.e., the nucleotide to be identified, may be a “single-nucleotide polymorphism” (SNP), which refers to any nucleotide sequence variation, preferably one that is common in a population of organisms and is inherited in a Medelian fashion. Typically, the SNP is either of two possible nucleotides, and there is little or no possibility of finding a third or fourth nucleotide identity at an SNP site.

The term “polymorphism” or “genetic variation,” as used herein, refers to the occurrence of two or more genetically determined alternative sequences or alleles at a defined position in a population. The allelic form occurring most frequently in a selected population is referred to as the wild type form. Other allelic forms are designated as variant forms. Diploid organisms may be homozygous or heterozygous for allelic forms.

The genetic variation may be associated with or cause diseases or disorders. The term “associated with,” as used herein, refers to the correlation between the occurrence of the genetic variation and the presence of a particular disease or a disorder. Such diseases or disorders may be human genetic diseases or disorders that include, but are not limited to, bladder carcinoma, colorectal tumors, sickle-cell anemia, thalassemias, a1-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease.

Target nucleic acids may be amplified before being combined with ODNPs as described below. Any known method for amplifying nucleic acids may be used. A few exemplary methods, including Qbeta Replicase, Strand Displacement Amplification, transcription-mediated amplification, RACE, and one-sided PCR, are summarized elsewhere herein.

2. Design of Oligonucleotide Primers (ODNPs)

Methods, kits and compositions of the present invention typically involve or include one or more ODNPs which generally contain a partial IRERS and a region of complementarity with a target nucleic acid. For the purpose of simplicity, the target nucleic acid is described as a single-stranded nucleic acid below. However, one of ordinary skill in the art would readily design the ODNP pair(s) of the present invention wherein the target nucleic acids are double-stranded.

The term “oligonucleotide” (ODN) refers to a nucleic acid fragment (typically DNA or RNA) obtained synthetically as by a conventional automated nucleic acid (e.g., DNA) synthesizer. Oligonucleotide is used synonymously with the term polynucleotide. The term “oligonucleotide primer” (ODNP) refers to any polymer having two or more nucleotides used in a hybridization, extension, and/or amplification reaction. The ODNP may be comprised of deoxyribonucleotides, ribonucleotides, or an analog of either. As used herein for hybridization, extension, and amplification reactions, ODNPs are generally between 8 and 200 bases in length. More preferred are ODNPs of between 12 and 50 bases in length and still more preferred are ODNPs of between 18 and 32 bases in length.

In one aspect, the present invention provides an ODNP useful for producing a portion of a target nucleic acid containing a nucleotide of interest at a defined position. The ODNP comprises an oligonucleotide sequence complementary to a nucleotide sequence of a target nucleic acid at a location 3′ to the defined position. The complementarity between the ODNP and its target need not be exact, but must be sufficient for the ODNP to selectively hybridize with the target, so that the ODNP is able to function as a primer for extension and/or amplification using the target as a template.

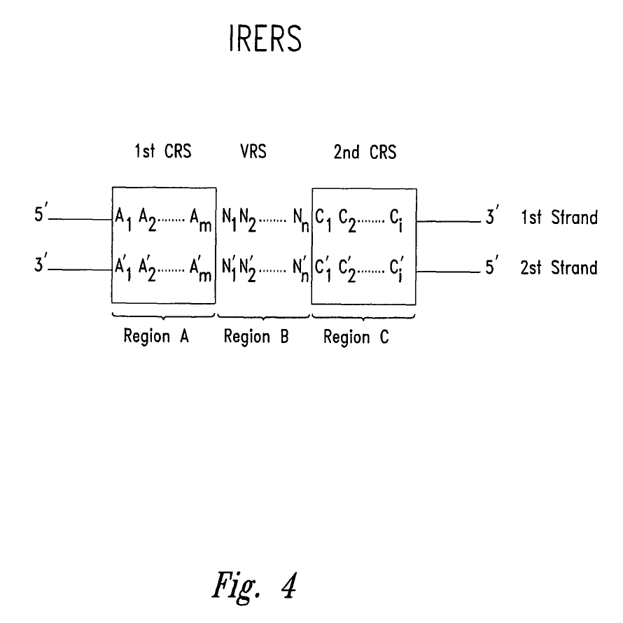

The ODNP further comprises a first CRS of a first strand of an IRERS at a location 3′ to the oligonucleotide sequence complementary to a portion of the target. As described in more detail below, a complete IRERS is a double-stranded oligonucleotide sequence comprising a first CRS and a second CRS linked with a VRS (FIG. 4). In addition, according to the present invention, the complete IRERS is selected so that digestion at the IRERS produces a 5′ overhang. Preferably, the 5′ overhang consists of a single nucleotide. The ODNP is so designed that when it anneals to the target, the distance between the nucleotide corresponding to the 3′ terminal nucleotide of the ODNP and the defined position in the target is within the range 0 to n-1 (preferably 0 to (n-1)/2) where n is the number of variable nucleotides in the IRERS. Such a design allows the extension product of the ODNP to incorporate a nucleotide complementary to the nucleotide of interest. In addition, the ODNP is further designed so that when typically used in combination with another ODNP to produce a fragment containing a complete IRERS and a nucleotides of interest (described in detail below), the nucleotide of interest is within both the VRS and the 5′ overhang produced by digestion at the IRERS.

In a preferred embodiment, the ODNP further comprises one or more nucleotides complementary to the target nucleic acid at the 3′ terminus of the first CRS. The presence of such nucleotides facilitates extension of the primer as the sequence of the first CRS in the ODNP may or may not be exactly complementary to the corresponding nucleotide sequence of the target. Also preferred is the ODNP with its 5′-terminus linked to a biotin molecule.

In another aspect, the present invention provides an ODNP pair for producing a portion of a target nucleic acid containing a nucleotide to be identified at a defined position (FIG. 3). One primer of the ODNP pair (“the first ODNP” or “the forward primer”) comprises a nucleic acid sequence complementary to a nucleotide sequence of a target nucleic acid at a location 3′ to the defined position (“the first region of the target nucleic acid”), whereas the other primer (“the second ODNP” or “the reverse primer”) comprises a nucleic acid sequence complementary to a nucleotide sequence of the complement of the target nucleic acid at a location 3′ to the complementary nucleotide of the nucleotide at the defined position (“the first region of the complement”). The complementarity between the ODNPs and their corresponding target nucleic acid, or the complement thereof, need not be exact, but must be sufficient for the ODNPs to selectively hybridize with the target nucleic acid, or the complement thereof, such that the ODNPs are able to function as primers for extension and/or amplification using the target nucleic acid, or the complement thereof, as a template. Generally, each ODNP contains at least 6, preferably 8, more preferably 10, most preferably 12, 14, or 16 nucleotides that are complementary to the target nucleic acid or the complement thereof. Because each ODNP of the ODNP pair hybridizes to a target nucleic acid, or the complement thereof, at a location 3′ to the defined position in the target or the complementary position in the complement of the target, the resulting extension and/or amplification products from the ODNP pair contains the nucleotide to be identified at the defined position.

Each ODNP in the ODNP pair of the present invention further comprises a partial IRERS, but not a complete IRERS, at a location 3′ to, or preferably at the 3′ terminus of, its nucleic acid sequence described above (i.e., the sequence complementary to the target nucleic acid or the complement thereof). Generally, the first ODNP and the second ODNP comprise the first CRS of the first strand of the IRERS and the second CRS of the second strand of the IRERS, respectively. In addition, the IRERS is selected so that digestion at the IRERS with a restriction endonuclease that recognizes the IRERS produces a 5′ overhang. Preferably, the 5′ overhang consists of a single nucleotide. Furthermore, the first ODNP and the second ODNP are so spaced that (1) the extension and/or amplification product with the ODNP pair as primers and the target nucleic acid as a template contains a complete IRERS. In other words, the number of nucleotides between the first and the second CRS is the exact number of nucleotides in the VRS so that the extension and/or amplification product from both ODNPs can be digested by a RE that recognizes the complete IRERS. Moreover, the nucleic acid to be identified in the extension and/or amplification product is within both the VRS and the 5′ overhang produced by digestion at the IRERS. The partial IRERS in each ODNP may or may not be complementary to the target nucleic acid.

In a preferred embodiment, each ODNP of the ODNP pair further contains one or more nucleotides that is complementary to the target nucleic acid or the complement thereof (“the second region of the target nucleic acid” and “the second region of the complement,” respectively) at a location 3′ to, or preferably the 3′ terminus of, the CRS. Such nucleotides are a portion of the VRS (FIG. 3). The number of the nucleotides between first and second regions of the target nucleic acid or the complement thereof may be larger or smaller, but preferably equal to, the number of nucleotides of ODNPs between their two regions that are complementary to the target nucleic acids or the complement thereof.

Also preferred is that one ODNP of the ODNP pair has its 5′ terminus linked to a biotin molecule. Such a linkage facilitates separation of a digestion product containing the ODNP from others before such a product is filled at its 3′ recessed terminus with a fluorescence labeled nucleoside triphosphate and subsequently characterized by fluorescence polarization as described in detail below.

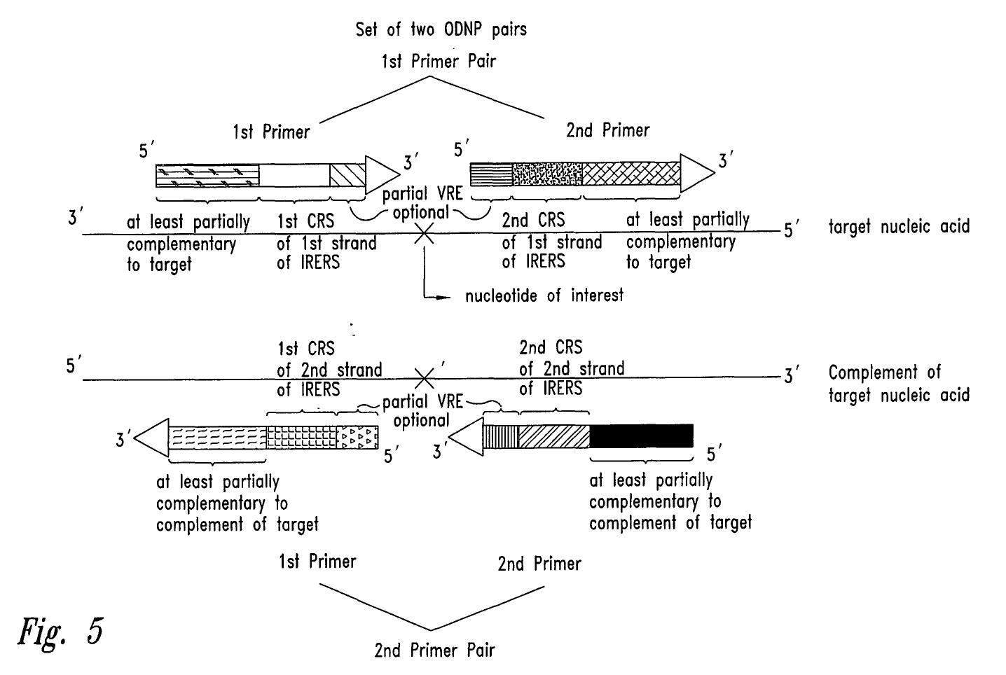

In another aspect, the present invention provides a set of two ODNP pairs for producing a portion of a target nucleic acid containing a nucleotide to be identified at a defined position (FIG. 5). Each pair of the set contain a first ODNP and a second ODNP. The first ODNP of the first ODNP pair comprises an oligonucleotide sequence complementary to a nucleotide sequence of the target nucleic acid at a location 3′ to the defined position. It further comprises a first CRS of a first strand of an IRERS at a location 3′ to, preferably at the 3′ terminus of, the above oligonucleotide sequence. The second ODNP of the first ODNP pair comprises an oligonucleotide sequence complementary to a nucleotide sequence of the target nucleic acid at a location 5′ to the defined position. It further comprises a second CRS of the first stand of the IRERS at a location 5′ to, preferably at the 5′ terminus, of the above oligonucleotide sequence. The first ODNP of the second ODNP pair comprises an oligonucleotide sequence complementary to a nucleotide sequence of the complement of the target nucleic acid at a location 5′ to the position in the complement corresponding to the defined position in the target nucleic acid. It further comprises the first CRS of the second strand of the IRERS at a location 5′ to, preferably at the 5′ terminus of, the above oligonucleotide sequence. The second ODNP of the second ODNP pair comprises an oligonucleotide sequence complementary to a nucleotide sequence of the complement of the target nucleic acid at a location 3′ to the position in the complement corresponding to the defined position in the target. It further comprises the sequence of the second CRS of the second strand of the IRERS at a location 3′ to, preferably at the 3′ terminus of, the above oligonucleotide sequence. The IRERS is selected to produce a 5′ overhang upon digestion. In addition, the set of two ODNP pairs are so spaced that the nucleotide to be identified in an extension and ligation fragment from such a set is within the 5′ overhang resulting from digestion at the IRERS.

In a preferred embodiment, the first ODNP of the first ODNP pair and the second ODNP of the second ODNP pair each further contains one or more nucleotides that are complementary to a nucleotide sequence of the target nucleic acid or the complement thereof at the 3′ terminus of the first or the second CRS. Such complementarity at the 3′ termini of the ODNPs increases the extension and/or amplification efficiency from the ODNPs.

General techniques for designing sequence-specific primers are well known. For instance, such techniques are described in books, such as PCR Protocols: Current Methods and Application edited by Bruce A. White, 1993; PCR Primer: A Laboratory Manual edited by Carl W. Dieffenbach and Gabriela S. Dveksler, 1995; PCR (Basics: From Background to Bench) by McPherson et al.; PCR Applications: Protocols for Functional Genomics edited by Michael A. Innis, 1999; PCR: Introduction to Biotechniques Series by Newton and Graham, 1997; PCR Protocols: A Guide to Methods and Applications by Gelfand et al., 1990; PCR Strategies by Michael A. Innis; PCR Technology: Current Innovations, by Griffin and Griffin, 1994; and PCR: Essential Techniques, edited by J. F. Burke. In addition, softwares for designing primers are also available, including Primer Master (see, Proutski and Holmes, Primer Master: A new program for the design and analysis of PCR primers. Comput. Appl. Biosci. 12: 253-5, 1996) and OLIGO Primer Analysis Software from Molecular Biology Insights, Inc. (Cascade, Colo., USA). The above reference books and description of softwares are incorporated herein by reference in their entireties. As mentioned previously, suitably designed primers may be synthesized by techniques known in the art, or more conveniently, may be purchased from one of the many supply houses that will prepare oligonucleotide primers having a custom sequence.

3. Nucleic Acid Hybridization and Extension/Amplification

Methods, kits and compositions of the present invention may involve or include ODNP that are hybridized to the target nucleic acid, where the ODNP facilitates the production and/or amplification of a defined nucleotide locus within the target nucleic acid. The ODNP and target nucleic acid are thus preferably combined under base-pairing condition. Selection of suitable nucleic acid hybridization and/or amplification conditions are available in the art by, e.g., reference to the following laboratory research manuals: Sambrook et al., “Molecular Cloning” (Cold Spring Harbor Press, 1989) and Ausubel et al., “Short Protocols in Molecular Biology” (1999) (incorporated herein by reference in their entirety).

Depending on the application envisioned, the artisan may vary conditions of hybridization to achieve desired degrees of selectivity of ODNP towards target sequence. For applications requiring high selectivity, relatively stringent conditions may be employed to form the hybrids, such as e.g., low salt and/or high temperature conditions, such as from about 0.02 M to about 0.15 M salt at temperatures of from about 50° C. to about 70° C. Such selective conditions are relatively intolerant of large mismatches between the ODNP target nucleic acid.

Alternatively, hybridization of the ODNPs may be achieved under moderately stringent buffer conditions such as, for example, in 10 mM Tris, pH 8.3; 50 mM KCl; 1.5 mM MgCl₂ at 60° C. which conditions permit the hybridization of ODNP comprising nucleotide mismatches with the target nucleic acid. The design of alternative hybridization conditions is well within the expertise of the skilled artisan.

After being hybridized to the target, the ODNPs are extended with the target or the complement thereof as a template using various methodologies known in the art, such as the polymerase chain reaction (PCR) and modified ligase chain reaction (LCR). For the purpose of convenient explanation, the hybridization and extension/amplification process will be described below for the situation where the target nucleic acid is single stranded. It is well within the skill of one of ordinary skill in the art to use an analogous process when the target nucleic acid is double stranded.

As illustrated in FIG. 1, which uses EcoN I as an exemplary restriction endonuclease, to obtain a portion of a target nucleic acid containing a defined nucleotide locus and a complete IRERS, at least three runs of extension reaction from the ODNP pair described above need be carried out. Briefly, the first run of extension is for the first ODNP having a first CRS to incorporate the complement of the nucleotide of interest in the first extension product. The second ODNP having a second CRS then hybridizes to and extends using the first extension product as a template and thereby incorporate the nucleotide of interest and the first CRS in a second extension product. An unextended first ODNP then hybridizes to and extends using the second extension product as a template and thereby form, in combination with the second extension product, a double-stranded nucleic acid fragment. Because the first ODNP and the second ODNP of the ODNP pair are spaced in a distance of the same number of base pairs as that of the VRS, the double-stranded nucleic acid fragment resulting from the three runs of extensions contains a complete IRERS.

While three runs of extension reactions are sufficient to produce a fragment containing a defined nucleotide locus within a target nucleic acid and a complete IRERS, preferably, more than three extension reactions are conducted to amplify the fragment. As one of ordinary skill in the art would appreciate, in the subsequent runs of extension, the first primer can hybridize to and extend using any of the target nucleic acid, the second extension product and the complement of the third extension product, as a template. Similarly, in the subsequent runs of extension, the second primer can hybridize to and extend using either the first extension product or the third extension product as a template. However, because the third extension product and the complement thereof are shorter than any of the target nucleic acid, the first extension product and the second extension product, they are the preferred templates for subsequent extension reactions from either the first or the second ODNPs. This is because the extension efficiency with a short fragment as a template is higher than that with a large fragment as a template. With the increase of the number of extension reactions, the double stranded fragment containing both the nucleotide to be identified and a complete IRERS accumulates quickly than other molecules in the reaction mixture. Such accumulation increases the sensitivity of subsequent characterization of the fragment after being digested with a RE that recognizes the complete IRERS.

The extension/amplification reaction can be carried out known in the art, including PCR methods. For instance, U.S. Pat. Nos. 4,683,195, 4,683,202 and 4,800,159 all describe PCR methods. In addition, PCR methods are also described in several books, e.g., Gelfand et al., “PCR Protocols: A Guide to Methods and Application” (1990); Burke (ed), “PCR: Essential Techniques”; McPherson et al. “PCR (Basic: From Background to Bench)”. Each of the above references is incorporated herein by reference in its entirety. Briefly, in PCR, two ODNPs are prepared that are complementary to regions on opposite complementary strands of the target nucleic acid sequence. An excess of deoxynucleoside triphosphates is added to a reaction mixture along with a DNA polymerase (e.g., Taq or Pfu polymerase). If the target nucleic acid sequence is present in a sample, the ODNPs will bind to the target and the polymerase will cause the ODNPs to be extended along the target nucleic acid sequence by adding on nucleotides. By raising and lowering the temperature of the reaction mixture, the extended ODNPs will dissociate from the target to form reaction products, excess ODNPs will bind to the target and to the reaction product and the process is repeated.

Exemplary PCR conditions according to the present invention may include, but are not limited to, the following: 100 μl PCR reactions comprise 100 ng target nucleic acid; 0.5 μM of each first ODNP and second ODNP; 10 mM Tris, pH 8.3; 50 mM KCl; 1.5 mM MgCl₂; 200 μM each dNTP; 4 units Taq™ DNA Polymerase (Boehringer Mannheim; Indianapolis, Ind.), and 880 ng TaqStart™ Antibody (Clontech, Palo Alto, Calif.). Exemplary thermocycling conditions may be as follows: 94° C. for 5 minutes initial denaturation; 45 cycles of 94° C. for 30 seconds, 60° C. for 30 seconds, 72° C. for 1 minute; final extension at 72° C. for 5 minutes. Exemplary nucleic acid polymerases may include one of the thermostable DNA polymerases that are readily available in the art such as, e.g., Taq™, Vent™ or PFU™. Depending on the particular application contemplated, it may be preferred to employ one of the nucleic acid polymerases having a defective 3′ to 5′ exonuclease activity.

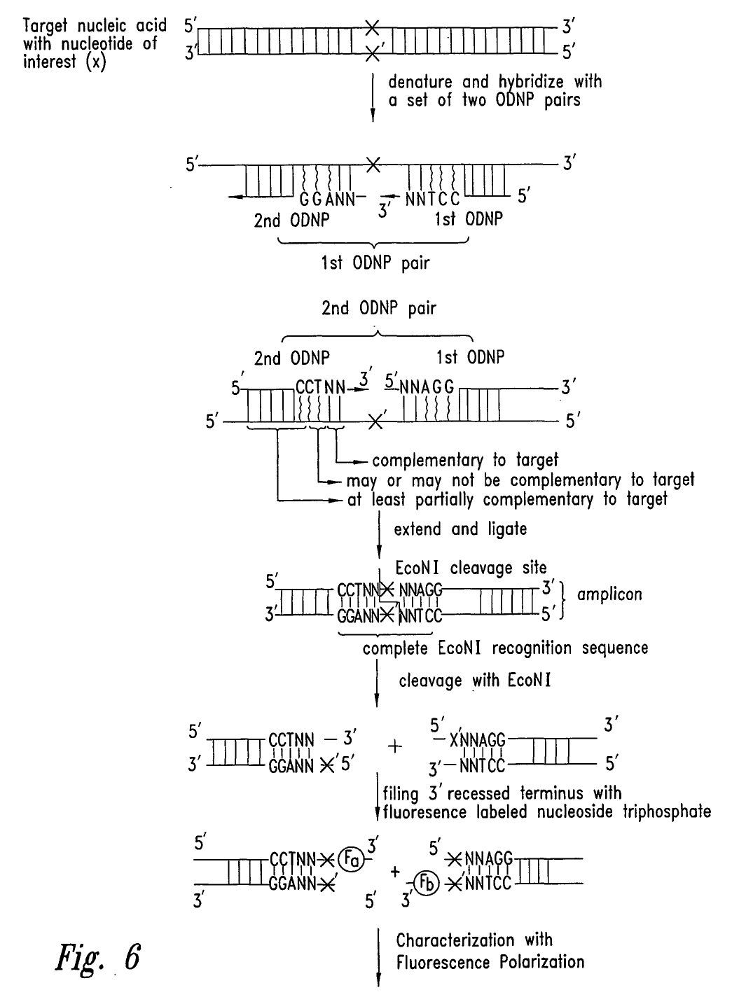

An alternative way to make and/or amplify a fragment containing a nucleotide to be identified and a complete IRERS is by a modified ligase chain reaction, referred to herein as the gap-LCR (Abravaya, et al., Nucleic Acids Res. 23:675-682 (1995)), using the set of two ODNP pairs described above (FIG. 6). Briefly, in the presence of the target sequence, each pair of the set will bind to the target, or the complement thereof, located 5′ and 3′ of (on either side of) the nucleotide of interest in the target nucleic acid. In the presence of a polymerase and a ligase, the gap between the two ODNPs of each pair will be filled in and the ODNPs, of each pair ligated to form a single unit. By temperature cycling, as in PCR, bound ligated units dissociate from the target and then serve as “target sequences” for ligation of excess ODNP pairs. Thus, LCR uses both a nucleic acid polymerase enzyme and a nucleic acid ligase enzyme to drive the reaction. Exemplary nucleic acid polymerases may include one of the thermostable DNA polymerases that are readily available in the art such as, e.g., Taq™, Vent™ or PFU™. Exemplary nucleic acid ligases may include T4 DNA ligase, or the thermostable Tsc or Pfu DNA ligases. U.S. Pat. No. 4,883,750, incorporated herein by reference in its entirety, describes an alternative method of amplification similar to LCR for binding ODNP pairs to a target sequence.

Exemplary gap-LCR conditions may include, but are not limited to, the following: 50 μl LCR reactions comprise 500 ng DNA; a buffer containing 50 mM EPPS, pH 7.8, 30 mM MgCl₂, 20 mM K⁺, 10 μM NAD, 1-10 μM gap filling nucleotides, 30 nM each oligonucleotide primer, 1 U Thermus flavus DNA polymerase, lacking 3′→5′ exonuclease activity (MBR, Milwaukee, Wis.), and 5000 U T. thermophilus DNA ligase (Abbott Laboratories). Cycling conditions may consist of a 30 s incubation at 85° C. and a 30 s incubation at 60° C. for 25 cycles and may be carried out in a standard PCR machine such as a Perkin Elmer 9600 thermocycler.

Another way to provide a double-stranded nucleic acid fragment containing a nucleotide of interest and a complete IRERS is by another modified ligase chain reaction, using two ODNPs and a single-stranded oligonucleotide. The first ODNP comprises an oligonucleotide sequence complementary to a nucleotide sequence of a target nucleic acid at a location 3′ to a nucleotide of interest in the target nucleic acid and a first CRSD of a first strand of an IRERS. The second ODNP comprises an oligonucleotide sequence complementary to a nucleotide sequence of the target at a location 5′ to the nucleotide of interest and a second CRS of the first strand of the IRERS. In the present of the target, a DNA polymerase and a DNA ligase, the two ODNPs extend and ligate with each other and the resulting product incorporates a nucleotide complementary to the nucleotide of interest in the target. Such a product is then annealed to a single-stranded oligonucleotide having a sequence complementary to the amplification and ligation product at least within the region from the 5′ terminus of the first ODNP and 3′ terminus of the second ODNP and a universal nucleotide at the position complementary to the nucleotide of interest.

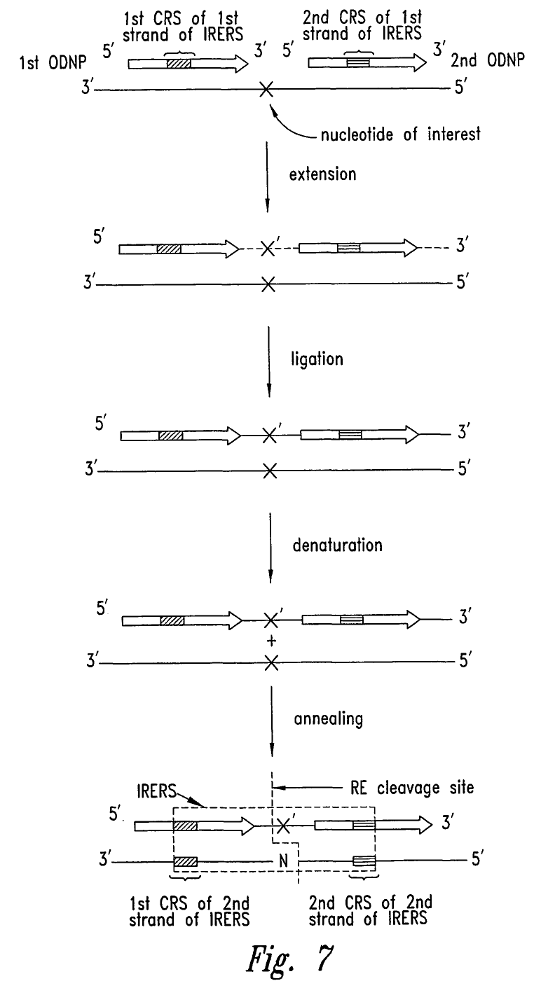

Another way to provide a double-stranded nucleic acid fragment containing a nucleotide to be identified at a defined location in a target nucleic acid and a complete IRERS is illustrated in FIG. 7. A primer pair is mixed with the target. One primer (“the first ODNP”) comprises an oligonucleotide sequence complementary to a nucleotide sequence of the target nucleic acid at a location 3′ to the defined position in the target and a first CRS of a first stand of an IRERS, whereas the other primer (“the second ODNP”) comprises an oligonucleotide sequence complementary to a nucleotide sequence of the target at a location 5′ to the defined position and a second CRS of the first strand of the IRERS. The two primers are then extended using the target as the template to incorporate the complement of the nucleotide to be identified (also referred to as “nucleotide of interest”). The extension products from the two primers are ligated and subsequently disassociated from the target. The disassociated, ligated extension product is then annealed to another nucleic acid molecule that contains the sequence complementary to the ligated extension product in the region from the 5′ terminus of the first ODNP to the 3′ terminus of the second ODNP. This nucleic acid molecule contains a universal nucleotide at a position corresponding to the complement of the nucleotide of interest in the ligated extension product. Such annealing produces a double stranded nucleic acid containing a complete IRERS and the complement of the nucleotide of interest. The primer pair is so spaced that the complement of the nucleotide of interest in the ligated extension product is within the VRS of the IRERS and the 5′ overhang produced by digestion at the IRERS.

In addition to the techniques described above, a number of other template dependent methodologies may be used either to amply target nucleic acids before combining the target nucleic acids with the ODNPs of the present invention. Alternatively, such methodologies may be used, in combination of the ODNP pair or the set of two ODNP pairs described above, to produce a fragment containing a portion of a target nucleic acid with a defined nucleotide locus and a complete IRERS. For instance, Qbeta Replicase, described in PCT Intl. Pat. Appl. Publ. No. PCT/US87/00880, incorporated herein by reference in its entirety, may alternatively be used with methods of the present invention. By this method, a replicative sequence of RNA that has a region complementary to that of a target is added to a sample in the presence of an RNA polymerase. The polymerase will copy the replicative sequence that can then be detected.

Alternatively, Strand Displacement Amplification (SDA) may be employed to achieve isothermal amplification of nucleic acids. By this methodology, multiple rounds of strand displacement and synthesis, i.e. nick translation, are utilized. A similar method, called Repair Chain Reaction (RCR) is another method of amplification which may be useful in the present invention and involves annealing several ODNPs throughout a region targeted for amplification, followed by a repair reaction in which only two of the four bases are present. The other two bases can be added as biotinylated derivatives for easy detection. A similar approach is used in SDA.

Other nucleic acid amplification procedures include transcription-based amplification systems (TAS) (also referred to as transcription-mediated amplification, or TMA) (Kwoh et al., 1989; PCT Intl. Pat. Appl. Publ. No. WO 88/10315, incorporated herein by reference in its entirety), including nucleic acid sequence based amplification (NASBA) and 3SR. In NASBA, the nucleic acids can be prepared for amplification by standard phenol/chloroform extraction, heat denaturation of a sample, treatment with lysis buffer and application onto minispin columns for isolation of DNA and RNA or guanidinium chloride extraction of RNA. These amplification techniques involve annealing an ODNP that has sequences specific to the target sequence. Following extension of the primer, DNA/RNA hybrids are digested with RNase H while double stranded DNA molecules are heat-denatured again. In either case the single stranded DNA is made fully double stranded by addition of a second target-specific ODNP, followed by polymerization. The double stranded DNA molecules are then multiply transcribed by a polymerase such as one of the RNA polymerases that are readily available in the art, e.g., SP6, T3, or T7. In an isothermal cyclic reaction, the RNAs are reverse transcribed into DNA, and transcribed once again with a polymerase such as T7 or SP6. The resulting products, whether truncated or complete, indicate target-specific sequences.

Eur. Pat. Appl. Publ. No. 329,822, incorporated herein by reference in its entirety, discloses a nucleic acid amplification process involving cyclically synthesizing single-stranded RNA (“ssRNA”), ssDNA, and double-stranded DNA (dsDNA), which may be used in accordance with the present invention. The ssRNA is a first template for a first ODNP, which is elongated by reverse transcriptase (RNA-dependent DNA polymerase). The RNA is then removed from the resulting DNA:RNA duplex by the action of ribonuclease H (RNase H, an RNase specific for RNA in a duplex with either DNA or RNA). The resultant ssDNA is a second template for a second ODNP, which also includes the sequences of an RNA polymerase promoter (exemplified by T7 RNA polymerase) 5′ to its homology to its template. This ODNP is then extended by DNA polymerase (exemplified by the large “Klenow” fragment of E. coli DNA polymerase I), resulting in a double-stranded DNA (“dsDNA”) molecule, having a sequence identical to that of the original RNA between the ODNPs and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

PCT Intl. Pat. Appl. Publ. No. WO 89/06700, incorporated herein by reference in its entirety, discloses a nucleic acid sequence amplification scheme based on the hybridization of a promoter/ODNP sequence to a target single-stranded DNA (“ssDNA”) followed by transcription of many RNA copies of the sequence. This scheme is not cyclic; since new templates are not produced from the resultant RNA transcripts. Other amplification methods include “RACE” (Frohman, 1990), and “one-sided PCR” (Ohara, 1989) which are well-known to those of skill in the art.

4. Restriction Endonucleases and Digestion Conditions

Methods, kits and compositions of the present invention typically involve or include one or more interrupted restriction endonucleases. The term “restriction endonuclease” (RE) refers to the class of nucleases that bind to unique double stranded nucleic acid sequences and that generate a cleavage in the double stranded nucleic acid that results in either blunt, double stranded ends, or single stranded ends with either a 5′ or a 3′ overhang.

As used herein, the term “interrupted restriction endonuclease recognition sequence” (IRERS) is defined as a restriction endonuclease recognition site that is comprised of a “first constant recognition sequence (CRS),” a “second CRS,” and a “variable recognition sequence (VRE)” that links the first and second CRSs (FIG. 4). According to the present invention, “first CRS” (also referred to as “Region A”) is defined as that region of the IRERS that contains the constant (not variable) nucleotides of the IRERS that are located 5′ of the VRE of the IRERS. “Second CRS” (also referred to as “Region C”) is defined as that region of the IRERS that contains the constant (not variable) nucleotides of the IRERS that are located 3′ of the VRE of the IRERS. According to the present invention, the “VRE” (also referred as “Region B”) is defined as the stretch of one or more variable nucleotides that are located between the first and second CRSs.

The term “EcoN I” refers to an exemplary RE that binds to, and cleaves, a unique double-stranded nucleic acid sequence shown below:

| | |
|---|---|
| 5′-CCTNN/NNNAGG-3′ | (SEQ ID NO. 1) |
| 3′-GGANNN/NNTCC-5′ | (SEQ ID NO. 2) |

wherein N designates an undefined nucleotide, that is, any one of the four nucleotides: A, T, G, and C. The bottom and top strands are cleaved 6 bases in from the 3′-OH ends (“/” indicates the cleavage sites). According to the present invention, nucleic acid fragments are produced that include this EcoN I binding sequence, where the nucleotide to be identified is positioned at the 6^{th} nucleotide (in bold face, also referred to as “the central nucleotide”) from the 5′ end of the top strand.

Any restriction endonuclease that recognizes an interrupted restriction endonuclease recognition sequence and produces a 5′ overhang containing one or more variable nucleotides upon digestion can be used in the present invention. Exemplary restriction endonucleases suitable for use in the present invention includes, but is not limited to, BlP I, BsaJ I, BssK I, BstE II, Bsu36 I, Dde I, EcoN I, Fnu4H I, Hinf I, Mae III, PflF I, Sau96 I, ScrF I and Tth111 I. Some of such enzymes are commercially available from numerous companies such as, e.g., New England Biolabs Inc. (Beverly, Mass.; www.neb.com); Stratagene (La Jolla, Calif.; www.stratagene.com), Promega (Madison, Wis.: www.promega.com), and Clontech (Palo Alto, Calif.; www.clontech.com). Non-commercially available restriction enzymes may be isolated and/or purified based on the teaching available in the art.

A nucleic acid fragment containing a portion of target nucleic acid with a defined nucleotide locus and a complete IRERS is digested (or cleaved) by a RE that recognizes the IRERS. Conditions for storage and use of restriction endonucleases used according to the present invention are readily available in the art, for example, by reference to one of the laboratory manuals such as Sambrook et al., supra and Ausubel et al., supra.

Briefly, the number of units of RE added to a reaction may be calculated and adjusted according to the varying cleavage rates of nucleic acid substrates. 1 unit of restriction endonuclease will digest 1 μg of substrate nucleic acid in a 50 μl reaction in 60 minutes. Generally, fragments (e.g., amplicons) may require more than 1 unit/μg to be cleaved completely. The restriction enzyme buffer is typically used at IX concentration in the reaction. Some restriction endonucleases require bovine serum albumin (BSA) (usually used at a final concentration of 100 μg/ml for optimal activity). Restriction endonucleases that do not require BSA for optimal activity are not adversely affected if BSA is present in the reaction.

Most restriction enzymes are stable when stored at −20° C. in the recommended storage buffer. Exposure to temperatures above −20° C. should be minimized whenever possible. All restriction endonucleases should be kept on ice when not otherwise being stored in the freezer. Enzymes should always be the last component added to a reaction.

The recommended incubation temperature for most restriction endonucleases is about 37° C. Restriction endonucleases isolated from thermophilic bacteria require higher incubation temperatures, typically ranging from 50° C. to 65° C. Incubation time may often be shortened if an excess of restriction endonuclease is added to the reaction. Longer incubation times are often used to allow a reaction to proceed to completion with fewer units of restriction endonuclease.

5. Incorporation of Fluorescence Labeled Nucleotides

Methods, kits and compositions of the present invention may involve or include incorporation of fluorescence labeled nucleotides into an EcoN I digestion product as described above. The incorporation of the labeled nucleotides facilitates the identification of a nucleotide of interest in a target nucleic acid.

As described above, an extension product or an amplicon using a target nucleic acid as a template and the ODNPs of the present invention as primers contains a complete IRERS. In addition, because the ODNPs are so designed in relation to the target nucleic acid that the nucleotide of interest is within both the VRS of the IRERS and a 5′ overhang produced by digestion at the IRERS. The 3′ recessed termini corresponding to the 5′ overhang may be subsequently filled in with fluorescence labeled nucleotides.

The incorporation of fluorescence labeled nucleotides into a 3′ recessed terminus of a digestion product may be catalyzed by a DNA polymerase in the presence of one or more fluorescent labeled deoxyribonucleoside triphosphates (dNTPs) or dideoxyribonucleoside triphosphates (ddNTPs). To increase the efficiency of the incorporation of fluorescence labeled nucleotides, alkaline phosphatase may be used to dephosphorylate all the existing non-labeled dNTPs from the extension/amplification reaction.

Preferably, the incorporation of fluorescence labeled nucleotides is carried out using an RNA polymerase in the presence of one or more fluorescence labeled ribonucleoside triphosphates (rNTPs). Because RNA polymerases have strict requirements for rNTPs and are unable to incorporate dNTPs, the need for a dephosphorylation step is abolished.

In a preferred embodiment, the ODNPs of the present invention are so designed in relation to a target nucleic acid that the nucleotide to be identified is within the VRS of an IRERS (e.g., EcoN I recognition site) and is the only nucleotide within a 5′ overhang produced by digestion at the IRERS (e.g., the central nucleotide with the EcoN I recognition site). Such an embodiment is particularly useful for identifying a nucleotide at a known SNP site. Typically, an SNP is either of two possible nucleotides and there is no possibility of finding a third or fourth nucleotide identity at an SNP site. If the two possible nucleotides are not complementary to each other (e.g., A and G), digestion products having 3′ recessed termini may be filled in using the two nucleotides each attached to a different fluorophore (e.g., BFL-ddATP and ROX-ddGTP). The signals from the two fluorophores attached to the nucleotides after the nucleotides are incorporated into the digestion products can be distinguished using fluorescence polarization described in detail below. The presence of both signals indicates that the biological sample from which the target nucleic acid is isolated is heterozygous at the SNP site. Alternatively, the digestion products may be divided into two portions: one portion filled in using a fluorophore linked to a first possible nucleotide, the other portion filled in using a fluorophore linked to a second possible nucleotide. Under this circumstance, the fluorophore linked to the first possible nucleotide may or may not be the same as that linked to the second possible nucleotide.

If the two possible nucleotides at an SNP site are complementary to each other, it may be necessary to separate digestion products from each other and then fill in one of the products using one or both possible nucleotides for the SNP site linked to fluorophores. The separation of the digestion products allows the distinction between the incorporation of the nucleotide at the SNP site from that of its complement nucleotide, which is the other possible nucleotide at the SNP site. For instance, assuming the two possible nucleotides at a particular SNP are A and T, the digestion of a nucleic acid fragment containing both the IRERS and the SNP by a restriction endonuclease that recognizes the IRERS produces two double-stranded oligonucleotides: one with a 5′ overhang consisting of the nucleotide “A,” the other with a 5′ overhang consisting of the nucleotide “T.” The two types of 5′ overhangs are present after digestion no matter whether the nucleotide at the SNP site is the nucleotide “A” or the nucleotide “T.” Without separation of the two digestion products, both fluorescence labeled nucleotide “A” and fluorescence labeled nucleotide “T” will be incorporated in a fill-in reaction, thus the identity of the nucleotide at the SNP site is not determined. However, if the two digestion products are separated from each other and each product is filled in with fluorescence labeled nucleotides individually, the identity of the nucleotide at the site can be determined.

Thus, the method of the present invention may be used to determine the identity of a nucleotide at a defined position in a target nucleic acid where the nucleotide can be any one of the four nucleotides (i.e., A, T or U, G and C). However, it may be necessary to separate digestion products from each other prior to a fill-in reaction.

The separation of digestion products from one another may be accomplished by any method suitable for separating small double-stranded oligonucleotides, including capillary electrophoresis (CE) and high-performance liquid chromatography (HPLC). Preferably, the first ODNP is linked to, at its 5′ terminus, a molecule that can be subsequently attached to a solid support (e.g., a biotin molecule) so the digestion product with a 5′ overhang consisting of a nucleotide of interest is also linked to this molecule via the first ODNP. This digestion product can be attached to a solid support (e.g., avidin- or streptavidin-coated beads) via this molecule and readily separable from the other digestion product (i.e., the product with a 5′ overhang consisting of the complement nucleotide of the nucleotide of interest) in solution.

Although the above description focuses on restriction endonucleases that produce 5′ overhangs consisting of single variable nucleotides, other restriction endonucleases that produce 5′ overhangs consisting of more than one nucleotides may also be used in the present application as long as the nucleotide to be identified is not identical, or complementary, to the other nucleotide(s) within the 5′ overhangs. If the nucleotide to be identified is not identical, or complementary, to the other nucleotide(s) within the 5′ overhangs (of which identities are known), the 3′ recessed termini corresponding to the 5′ overhangs may be filled in with unlabeled nucleoside triphosphate(s) at position(s) corresponding to those other than that of the nucleotide of interest, and with fluorescence labeled nucleoside triphosphate(s) at the position corresponding to that of the nucleotide of interest. For instance, if an amplification product using a target nucleic acid with a SNP at a defined position contains a recognition sequence for BssK I (i.e., /CCNGG where “/” indicates the cleavage site) and the SNP is the ‘N’ of the BssK I recognition sequence and may be either an “A” or a “T,” the two double strand digestion products may be first separated from each other and filled in with unlabeled cytidine triphosphate, unlabeled guanosine triphosphate, and fluorescence labeled adenosine triphosphate or deoxythymidine triphosphate.

Various fluorescent dyes or probes may be employed in the present invention. Fluorescent dyes are identified and quantified most directly by their absorption and fluorescence emission wavelengths and intensities. Emission spectra (fluorescence and phosphorescence) are much more sensitive and specific than absorption spectra. Other photophysical characteristics (like fluorescence anisotropy) are less widely used. The useful intensity parameters are quantum yield (QY) for fluorescence, and the molar extinction coefficient (ε) for absorption. QY is a measure of the total photon emission over the entire fluorescence spectral profile and the value of ε is specified at a given wavelength (usually the absorption maximum of the probe). A narrow optical bandwidth (<25 nm) is usually used for fluorescence excitation (via absorption), whereas the fluorescence detection bandwidth is more variable, ranging from full spectrum for maximal sensitivity to narrow band (˜20 nm) for maximal resolution. Fluorescence intensity per probe molecule is proportional to the product of ε and QY. Commercially important and exemplary fluorochromes that are widely used are fluorescein, tetramethylrhodamine, lissamine, Texas Red and BODIPYs.

Fluorescent labels are now commonly used for the detection of small nucleic acid fragments that have been separated by CE and HPLC. One group of labels that may be employed for this purpose are those based on near-infrared (near-IR) fluorescent dyes. In aqueous solution, these types of tags have a maximum absorption of light at >680 nm, followed by the emission of fluorescence at near-IR wavelengths (emission maximum, >700 nm). One advantage of using this type of fluorescence for detection is that it occurs in a spectral region where there is relatively little absorption or emission due to other compounds that might be present in biological samples. This, plus the fact that most near IR probes can be excited with commercially available lasers, provides this approach with low background signals and limits of detection that extend into the attomole range.

One of the limitations of fluorescence spectroscopy is the phenomenon of autofluorescence. One method to avoid autofluorescence is to employ fluorochromes that possess significantly longer delay times to emission (see Fernandes for review). These fluorochromes are usually luminescent metal chelates that are attached at the 5′-end of an ODN probe or primer.

Terbium deoxynucleoside triphosphates are available that allow the incorporation of time-resolved fluorochromes into “natural” nucleic acids. These probes have the advantage of the large Stokes shift, narrow emission bands and long lifetimes. Time-resolved fluorescence spectroscopy is particularly useful in structural biology and is used to monitor molecular interactions and motions that occur in the picosecond-nanosecond time range. Time-resolved fluorescence spectroscopy is beginning to dominate the analysis of biomolecular structure and dynamics.

Deoxyribonucleoside analogs that may be incorporated into a small nucleic acid fragment of the present invention, to thereby afford an effective characterization means for the small nucleic acid, include but are not limited to: Fluorescein-12-dUTP, Coumarin-5-dUTP, Tetramethylrhodamine-6-dUTP, Texas Red®-5-dUTP, Napthofluorescein-5-dUTP, Fluorescein Chlorotriazinyl-4-dUTP, Pyrene-8-dUTP, Diethylaminocoumarin-5-dUTP, Cyanine 3-dUTP, Cyanine 5-dUTP, Coumarin-5-dCTP, Fluorescein-12-dCTP, Tetramethylrhodamine-6-dCTP, Texas Red®-5-dCTP, Lissamine™-5-dCTP, Napthofluorescein-5-dCTP, Fluorescein Chlorotriazinyl-4-dCTP, Pyrene-8-dCTP, Diethylaminocoumarin-5-dCTP, Cyanine 3-dCTP, Cyanine 5-dCTP, Coumarin-5-dATP, Diethylaminocoumarin-5-dATP, Fluorescein-12-dATP, Fluorescein Chlorotriazinyl-4-dATP, Lissamine™-5-dATP, Napthofluorescein-5-dATP, Pyrene-8-dATP, Tetramethylrhodamine-6-dATP, Texas Red®-5-dATP, Cyanine 3-dATP, Cyanine 5-dATP, Coumarin-5-dGTP, Fluorescein-12-dGTP, Tetramethylrhodamine-6-dGTP, Texas Red-5-dGTP, and Lissamine™-5-dGTP.

Ribonucleoside analogs include but are not limited to: Fluorescein-12-UTP, Coumarin-5-UTP, Tetramethylrhodamine-6-UTP, Texas Red®-5-UTP, Lissamine™-5-UTP, Napthofluorescein-5-UTP, Fluorescein Chlorotriazinyl-4-UTP, Pyrene-8-UTP, Cyanine 3-UTP, Cyanine 5-UTP, Coumarin-5-CTP, Fluorescein-12-CTP, Tetramethylrhodamine-6-CTP, Texas Red-5-CTP. Lissamine™-5-CTP, Napthofluorescein-5-CTP, Fluorescein Chlorotriazinyl-4-CTP, Pyrene-8-CTP, Cyanine 3-CTP, Cyanine 5-CTP, Coumarin-5-ATP, Fluorescein-12-ATP, Tetramethylrhodamine-6-ATP, Texas Red®-5-ATP, Lissamine™-5-ATP, Coumarin-5-GTP, Fluorescein-12-GTP, Tetramethylrhodamine-6-GTP, Texas Red®-5-GTP, and Lissamine™-5-GTP.

Dideoxy analogs include but are not limited to: Fluorescein-12-ddUTP, FAM-ddUTP, ROX-ddUTP, R6G-ddUTP, TAMRA-ddUTP, JOE-ddUTP, R110-ddUTP, Fluorescein-12-ddCTP, FAM-ddCTP, ROX-ddCTP, R6G-ddCTP, TAMRA-ddCTP, JOE-ddCTP, R110-ddCTP, Fluorescein-12-ddGTP, FAM-ddGTP, ROX-ddGTP, R6G-ddGTP, TAMRA-ddGTP, JOE-ddGTP, R110-ddGTP, Fluorescein-12-ddATP, FAM-ddATP, ROX-ddATP, R6G-ddATP, TAMRA-ddATP, JOE-ddATP, and R110-ddATP.

All of the above analogs can be radiolabeled with ³H, deuterium, ³²P, ¹⁴C, ³⁵S and other radioisotopes.

Analogs can also be un-natural nucleoside analogs including, but not limited to, the following: 8-Bromo-2′-deoxyadenosine-TTP, 8-Oxo-2′-deoxyadenosine, Etheno-2′-deoxyadenosine-TTP, Etheno-2′-deoxyadenosine-TTP, N⁶-Methyl-2′-deoxyadenosine-TTP, 2,6-Diaminopurine-2′-deoxyriboside-TTP, 8-Bromo-2′-deoxyguanosine-TTP, 7-Deaza-2′-deoxyguanosine-TTP, 2′-Deoxyisoguanosine-TTP, -Oxo-2′-deoxyguanosine-TTP, O⁶-Methyl-2′-deoxyguanosine-TTP, S⁶-DNP-2′-deoxythioguanosine-TTP, 3-Nitropyrrole-2′-deoxyriboside-TTP, 5-Propynyl-2′-deoxyuridine-TTP, 5-Fluoro-2′-deoxyuridine-TTP, 2′-deoxyuridine-TTP, 5-Bromo-2′-deoxyuridine-TTP, 5-Iodo-2′-deoxyuridine-TTP, and 4-Triazolyl-2′-deoxyuridine-TTP.

6. Fluorescence Polarization (FP)

FP is based on the property that when a fluorescent molecule is excited by plane-polarized light, it emits polarized fluorescent light into a fixed plane if the tagged-molecules do not significantly rotate between excitation and emission. If the molecule is small enough and rotates and tumbles in space, however, fluorescence polarization is not observed fully by the detector.

The fluorescence polarization of a molecule is proportional to the molecule's rotational relaxation time (usually the time it takes to rotate through an angle of 68.5°), which is related to properties of the solution such as the viscosity, temperature, and molecular volume of the analyte or biomolecule. Therefore, if the viscosity and temperature are held constant, fluorescence polarization is directly proportional to molecular volume, which, in turn, is directly proportional to molecular weight. Larger tagged molecules rotate and tumble slowly in space and, accordingly, fluorescence polarization values can be obtained. In contrast, smaller molecules rotate and tumble faster and fluorescence polarization cannot be measured.

The present invention uses fluorescence polarization to detect fluorescence labeled nucleotides upon their incorporation into double-stranded oligonucleotides (i.e., EcoN I digestion products) and thus determines the identity of the nucleotide of interest. Fluorescence labeled nucleoside triphosphates are small and rotate rapidly in solution. Thus, absent incorporation into a small nucleic acid fragment, fluorescent-tagged-nucleoside triphosphates are undetectable by fluorescence polarization. The fill-in reaction described above that incorporates a fluorescence labeled nucleoside triphosphate into a nucleic acid fragment increases by about 20-fold the molecular weight of the fluorophore. Because of the increase in the molecular weight, the fluorescence polarization of the oligonucleotide can be detected and measured.

Currently, polarizing fluorometers and more than 50 fluorescence polarization immunoassays (FPIAs) are commercially available, many of which are routinely used in clinical laboratories for the measurement of therapeutics, metabolites, and drugs of abuse in biological fluids. (See, e.g., Checovich et al., Nature 375:254-256 (1995); published erratum appears in Nature 375:520 (1995) both of which are incorporated herein by reference in their entirety). Many polarizing fluorometers and FPIAs are capable of measuring samples in mitrotiter plates, thus the identity of nucleotides at defined position in target nucleic acids can be determined in parallel.

7. Software for Analysis of Sequence Information Derived from Fluorescence Polarization Detection

The method of the present invention may optionally comprises the use of one or more computer algorithms for analyzing the derived sequence information. Algorithms of the present invention may be encompassed within software packages that convert a detection signal, such as a fluorescence value of a given small nucleic acid fragment, to a genotyping call.

A genotyping system for use with a fluorescence based detection system may comprise one or more components as follows: a peak identification algorithm which identifies peaks above a certain threshold of intensity (area under the curve), an algorithm that identifies and records the retention time of the peaks between certain time intervals (e.g., between 1.75 and 3 minutes in a 5 minute run), an algorithm that calculates the intensity of peaks by measuring the area under the curve, an algorithm that calculates the number of peaks between a certain time interval, an algorithm that calculates the ratio of each set of two peaks, and an algorithm that calculates the allele calling from the ratiometric values. The software package and algorithms record the sample identification (sample ID), source, primer name and sequence, length of expected fragment, estimation of expected retention time, chromatography details, sample plate ID, sample well ID, date and time, number of peaks observed, observed retention times, and calculated allele call. The algorithms will also download the data to existed databases and check for accuracy of recording.

The software package that converts the presence of a label on the fragment to a genotyping call is composed of the following: an algorithm that calculates the allele calling from the ratiometric values of fluorescence or fluorescence polarization. The software package and algorithms record the sample identification (sample ID), source, primer name and sequence, mass to charge ratio of expected fragment, estimation of expected fluorescence ratios, instrument details, sample plate ID, sample well ID, date and time, and calculated allele call. The algorithms will also download the data to existing databases and check for accuracy of recording.

C. Applications for the Methods, Compositions and Compounds of the Present Invention

As discussed in detail herein above, the present invention provides methodology for identifying nucleotides (including identifying mutations and/or SNPs) at defined nucleotide loci within target nucleic acids. Methods according to the present invention will find utility in a wide variety of applications, including but not limited to genetic analysis for hereditary diseases, tumor diagnosis, disease predisposition, forensics or paternity, crop cultivation and animal breeding, expression profiling of cell function and/or disease marker genes, and identification and/or characterization of infectious organisms that cause infectious diseases in plants or animals and/or that are related to food safety. Described below are certain exemplary applications of the present invention.

1. Expression Profiling

Most mRNAs are transcribed from single copy sequences. Another property of cDNAs is that they represent a longer region of the genome because of the introns present in the chromosomal version of most genes. The representation varies from one gene to another but can be very significant as many genes cover more than 100 kb in genromic DNA, represented in a single cDNA. One possible use of molecular profiling is the use of probes from one species to find clones made from another species. Sequence divergence between the mRNAs of mouse and man permits specific cross-reassociation of long sequences, but except for the most highly conserved regions, prevents cross-hybridization of PCR primers.

Differential screening in complex biological samples such as developing nervous system using cDNA probes prepared from single cells is now possible due to the development of PCR-based and cRNA-based amplification techniques. Several groups reported previously the generation of cDNA libraries from small amounts of poly (A)+RNA (1 ng or less) prepared from 10-50 cells (Belyav et al., Nuc. Acids Res. 17:2919, 1989). Although the libraries were sufficiently representative of mRNA complexity, the average cDNA insert size of these libraries was quite small (<2 kb).

More recently, methodologies have been combined to generate both PCR-based (Lambolez et al., Neuron 9:247, 1992) and cRNA-based (Van Gelder et al., Proc. Natl. Acad. Sci. USA 87:1663, 1990) probes from single cells. After electrical recordings, the cytoplasmic contents of a single cell were aspirated with patch-clamp microelectrodes for in situ cDNA synthesis and amplification. PCR was used to amplify cDNA of selective glutamate receptor mRNAs from single Purkinje cells and GFAP mRNA from single glia in organotypic cerebellar culture (Lambolez et al., Neuron 9:247, 1992). In the case of cRNA amplification, transcription promoter sequences were designed into primers for cDNA synthesis and complex antisense cRNAs were generated by in vitro transcription with bacteriophage RNA polymerases.

The method of the present invention is useful for determining whether a particular cDNA molecule is present in cDNAs from a biological sample and further determine whether genetic variation(s) exist in the cDNA molecule.

2. Forensics

The identification of individuals at the level of DNA sequence variation offers a number of practical advantages over such conventional criteria as fingerprints, blood type, or physical characteristics. In contrast to most phenotypic markers, DNA analysis readily permits the deduction of relatedness between individuals such as is required in paternity testing. Genetic analysis has proven highly useful in bone marrow transplantation, where it is necessary to distinguish between closely related donor and recipient cells. Two types of probes are now in use for DNA fingerprinting by DNA blots. Polymorphic minisatellite DNA probes identify multiple DNA sequences, each present in variable forms in different individuals, thus generating patterns that are complex and highly variable between individuals. VNTR probes identify single sequences in the genome, but these sequences may be present in up to 30 different forms in the human population as distinguished by the size of the identified fragments. The probability that unrelated individuals will have identical hybridization patterns for multiple VNTR or minisatellite probes is very low. Much less tissue than that required for DNA blots, even single hairs, provides sufficient DNA for a PCR-based analysis of genetic markers. Also, partially degraded tissue may be used for analysis since only small DNA fragments are needed. The methods of the present invention are useful in characterizing polymorphism of sample DNAs, therefore useful in forensic DNA analyses. For example, the analysis of 22 separate gene sequences in a sample, each one present in two different forms in the population, could generate 1010 different outcomes, permitting the unique identification of human individuals.

3. Tumor Diagnostics

The detection of viral or cellular oncogenes is another important field of application of nucleic acid diagnostics. Viral oncogenes (v-oncogenes) are transmitted by retroviruses while their cellular counterparts (c-oncogenes) are already present in normal cells. The cellular oncogenes can, however, be activated by specific modifications such as point mutations (as in the c-K-ras oncogene in bladder carcinoma and in colorectal tumors). Each of the activation processes leads, in conjunction with additional degenerative processes, to an increased and uncontrolled cell growth. In addition, mutations may also inactivate the so-called “recessive oncogenes” and thereby leads to the formation of a tumor (as in the retinoblastoma (Rb) gene and the osteosarcoma). Accordingly, the present invention is useful in detecting or identifying mutations that activate oncogenes or inactivate recessive oncogenes, which in turn, cause cancers.

4. Transplantation Analyses

The rejection reaction of transplanted tissue is decisively controlled by a specific class of histocompatibility antigens (HLA). They are expressed on the surface of antigen-presenting blood cells, e.g., macrophages. The complex between the HLA and the foreign antigen is recognized by T-helper cells through corresponding T-cell receptors on the cell surface. The interaction between HLA, antigen and T-cell receptor triggers a complex defense reaction which leads to a cascade-like immune response on the body.

The recognition of different foreign antigens is mediated by variable, antigen-specific regions of the T-cell receptor—analogous to the antibody reaction. In a graft rejection, the T-cells expressing a specific T-cell receptor which fits to the foreign antigen, could therefore be eliminated from the T-cell pool. Such analyses are possible by the identification of antigen-specific variable DNA sequences which are amplified by PCR and hence selectively increased. The specific amplification reaction permits the single cell-specific identification of a specific T-cell receptor.

Similar analyses are presently performed for the identification of auto-immune disease like juvenile diabetes, arteriosclerosis, multiple sclerosis, rheumatoid arthritis, or encephalomyelitis.

Accordingly, the present invention is useful for determining gene variations in T-cell receptor genes encoding variable, antigen-specific regions that are involved in the recognition of various foreign antigens.

5. Genome Diagnostics

Four percent of all newborns are born with genetic defects; of the 3,500 hereditary diseases described which are caused by the modification of only a single gene, the primary molecular defects are only known for about 400 of them.

Hereditary diseases have long since been diagnosed by phenotypic analyses (anamneses, e.g., deficiency of blood: thalassemias), chromosome analyses (karyotype, e.g., mongolism: trisomy 21) or gene product analyses (modified proteins, e.g., phenylketonuria: deficiency of the phenylalanine hydroxylase enzyme resulting in enhanced levels of phenylpyruvic acid). The additional use of nucleic acid detection methods considerably increases the range of genome diagnostics.

In the case of certain genetic diseases, the modification of just one of the two alleles is sufficient for disease (dominantly transmitted monogenic defects); in many cases, both alleles must be modified (recessively transmitted monogenic defects). In a third type of genetic defect, the outbreak of the disease is not only determined by the gene modification but also by factors such as eating habits (in the case of diabetes or arteriosclerosis) or the lifestyle (in the case of cancer). Very frequently, these diseases occur in advanced age. Diseases such as schizophrenia, manic depression or epilepsy should also be mentioned in this context; it is under investigation if the outbreak of the disease in these cases is dependent upon environmental factors as well as on the modification of several genes in different chromosome locations.

Using direct and indirect DNA analysis, the diagnosis of a series of genetic diseases has become possible: bladder carcinoma, colorectal tumors, sickle-cell anemia, thalassemias, a1-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease. The present application is useful in diagnosis of any genetic diseases that are caused by point mutations at defined positions.

6. Infectious Disease

The application of recombinant DNA methods for diagnosis of infectious diseases has been most extensively explored for viral infections where current methods are cumbersome and results are delayed. In situ hybridization of tissues or cultured cells has made diagnosis of acute and chronic herpes infection possible. Fresh and fomalin-fixed tissues have been reported to be suitable for detection of papillomavirus in invasive cervical carcinoma and in the detection of HIV, while cultured cells have been used for the detection of cytomegalovirus and Epstein-Barr virus. The application of recombinant DNA methods to the diagnosis of microbial diseases has the potential to replace current microbial growth methods if cost-effectiveness, speed, and precision requirements can be met. Clinical situations where recombinant DNA procedures have begun to be applied include the identification of penicillin-resistant Neisseria gonorrhoeae by the presence of a transposon, the fastidiously growing chliamydia, microbes in foods; and simple means of following the spread of an infection through a population. The worldwide epidemiological challenge of diseases involving such parasites as leishmania and plasmodia is already being met by recombinant methods.

The present invention is useful to detect and/or measure genetic variations that are involved in infectious diseases, especially those in drug resistance genes. Thus, the present invention facilitates the characterization and classification of organisms that cause infectious diseases and consequently the treatment of such diseases caused by these organisms.

The following experimental example is offered by way of illustration, not limitation.

### EXAMPLE

### Genotype Assignment Using Fluorescence Polarization-ECON I Assay

This example discloses the use of fluorescence polarization (FP) and the EcoN I template-directed primer extension (fill-in) assay in assigning genotype.

Enzymes

EcoN I was obtained from New England BioLabs (Beverly Mass.), AmpliTaq and AmpliTaq-FS DNA polymerase were obtained from Perkin-Elmer Applied Biosystems Division (Foster City, Calif.).

Oligonucleotides

Oligonucleotides used are listed in Table 1. Four synthetic 48-mers with identical sequence except for position 23 were prepared (CF508-48), the variant bases are shown as boldface letters. PCR and EcoN I primers and synthetic template oligonucleotides were obtained from Life Technologies (Grand Island, N.Y.).

Dye-Labeled Dideoxyribonucleoside Triphosphates

Dideoxyribonucleoside triphosphates labeled with FAM, ROX, TMR, BFL, and BTR were obtained from NEN Life Science Products, Inc. (Boston, Mass.). Unlabeled ddNTPs were purchased from Pharmacia Biotech (Piscataway, N.J.).

PCR Amplification

Human genomic DNA (20 ng) from 34 unrelated individuals and 6 negative controls (water-blanks) were amplified for the marker D18S8 in 20-μl reaction mixtures containing 10 mM Tris-HCl (pH 8.3), 50 mM KCl, 1.5 mM MgCl₂, 0.2 mM DNTP, 1 μM of each primer, and AmpliTaq DNA polymerase (1 unit). The reaction mixture was held at 94° C. for 2 min followed by 10 cycles of 94° C. for 10 sec, ramping to 60° C. >90 sec, held at 60° C. for 30 sec, followed by 30 cycles of 94° C. for 10 sec, and 53° C. for 30 sec. For hemochromatosis mutation C282Y, 42 samples and 6 negative controls were amplified in the same buffer with these cycling conditions: 94° C. for 2 min followed by 10 cycles of 94° C. for 10 sec, ramping to 68° C. >90 sec, held at 68° C. for 30 sec, followed by 30 cycles of 94° C. for 10 sec, and 62° C. for 30 sec. At the end of the reaction, the reaction mixtures were held at 4° C. until further use.

Primer and dNTP Degradation

At the end of the PCR assay, 10 μl of an enzymatic cocktail containing shrimp alkaline phosphatase (2 units), E. coli exonuclease I (1 unit) in shrimp alkaline phosphatase buffer [20 mM Tris-HCl (pH 8.0), 10 mM MgCl₂] was added to the PCR product. The mixture was incubated at 37° C. for 30 min before the enzymes were heat inactivated at 95° C. for 15 min. The DNA mixture was kept at 4° C. and used in the FP-EcoN I assay without further quantification or characterization.

Primer Extension

To the PCR product was added 10 μl of 10× EcoN I buffer reaction mixture containing the EcoN I buffer and enzyme [100 mM Tris-HCl (pH 7.5), 50 mM NaCl, 10 mM MgCl₂, 0.025% Triton X-100, and 4 units of enzyme per genotype], 25 nM of each allele-specific dye-labeled ddNTP, 100 nM unlabeled other two ddNTPs, and AmpliTaq DNA polymerase FS (1 unit). The reaction mixtures were incubated at 93° 1 min, followed by 35 cycles of 93° 10 sec and 55° 30 sec. At the end of the reaction, the samples were held at 4° C.

Fluorescence Polarization Measurement

After the primer extension reaction, 100 μl of EcoN I buffer and 50 μl of methanol were added to each tube before they were transferred to a microtiter plate for FP measurement on a Fluorolite FPM2 instrument (Jolley Consulting and Research, Grayslake, Ill.) or Analyst fluorescence reader (LJL Biosystems, Sunnyvale, Calif.). Fluorescence polarization value was calculated using the formula:

P=[Ivv−Ivh]/[Ivv+Ivh]

where Ivv is the emission intensity measured when the excitation and emission polarizers are parallel and Ivh is the emission intensity measured when the emission and excitation polarizers are oriented perpendicular to each other. The degree of polarization is expressed by the unit mP, or a 0.001 ratio between (Ivv−Ivh) and (Ivv+Ivh).

Genotype Assignment

The average FP value and standard deviation of the negative control samples were determined for each set of experiment. The FP value of the test sample reactions was then compared to the average FP value of the control samples. If the net change is >40 mP (more than seven times the standard deviation of the controls), the test sample is scored as positive for the allele.

Although dye-labeled dideoxy-terminators have been used extensively in sequencing reactions and the sensitivity and specificity of template-directed primer extension genotyping methods are well established, the use of FP as a detection method in a fill-in reaction has not been reported before this work was done. Three sets of experiments were performed to show that FP is a simple, highly sensitive and specific detection method in a fill-in reaction for measuring SNPs. In the first set of experiments, four synthetic oligonucleotide templates containing the four possible nucleotides at one particular site in the middle of otherwise identical sequence were used to establish the sensitivity and specificity of FP detection of labeled nucleoside incorporation. In the second set of experiments, several dyes were tested for their utility in the fill-in assay. In the third set of experiments, PCR products were used as templates in a dual-color FP assay to show that accurate genotyping data could be obtained for both alleles of a marker or mutation in a homogeneous assay.

TABLE 1
Synthetic Templates and Primers Used in the FP Studies

| Oligo- | |
|---|---|
| nucleo- | |
| tides | |
| Syn- | |
| thetic | |
| tem- | |
| plates | Sequence (5′ to 3′) |
| CF508- | A T A T T C A T C A T A G G A A A C C T C A A A G A G G A T A T T T T C T T T A A T G G T G C C |
| 48A (SEQ | |
| ID NO. | |
| 3) | |
| CF508- | A T A T T C A T C A T A G G A A A C C T C A C A G A G G A T A T T T T C T T T A A T G G T G C C |
| 48C (SEQ | |
| ID NO. | |
| 4) | |
| CF508- | A T A T T C A T C A T A G G A A A C C T C A G A G A G G A T A T T T T C T T T A A T G G T G C C |
| 48G (SEQ | |
| ID NO. | |
| 5) | |
| CF508- | A T A T T C A T C A T A G G A A A C C T C A T A G A G G A T A T T T T C T T T A A T G G T G C C |
| 48T PCR | |
| primers | |
| (SEQ ID | |
| NO. 6) | |
| C282Y-p1 | T G G C A A G G G T A A A C A G A T C C |
| (SEQ ID | |
| NO. 7) | |
| C282Y-p2 | C T C A G G C A C T C C T C T C A A C C |
| (SEQ ID | |
| NO. 8) | |
| D1858-p1 | T T G C A C C A T G C T G A A G A T T G T |
| (SEQ ID | |
| NO. 9) | |
| D1858-p2 | A C C C T C C C C C T G A T G A C T T A |
| (SEQ ID | |
| NO. 10) | |

For each synthetic template (Table 1), one of the four possible bases was found at position 23. The synthetic 48-mers served as templates in four separate reactions where each was incubated with EcoN I and one of the four 5-carboxy-fluorescein (FAM)-labeled terminators in the presence of AmpliTaq DNA polymerase FS. At the end of the EcoN I reaction, the reaction mixture was diluted, the digestion products were separated via biotin molecules linked to one of the primers used by biotin-streptavidin interaction, and the fluorescence polarization of the separated digestion products was measured. Table 2 shows the results of these experiments.

TABLE 2
FP-TDI Assay with Synthetic Templates
Using FAM-Labeled Dye Terminators

| | FAM-ddA | FAM-ddC | FAM-ddG | FAM-ddU |
|---|---|---|---|---|
| Templates | (mP)^{a} | (mP)^{a} | (mP)^{a} | (mP)^{a} |
| CF508-48A | 52 | 36 | 54 | 89 |
| | 55 | 37 | 41 | 92 |
| | 52 | 39 | 48 | 101 |
| | 50 | 39 | 40 | 93 |
| CF508-48C | 57 | 37 | 121 | 39 |
| | 50 | 37 | 126 | 30 |
| | 55 | 39 | 115 | 40 |
| | 52 | 34 | 117 | 40 |
| CF508-48G | 52 | 92 | 42 | 42 |
| | 63 | 85 | 35 | 32 |
| | 50 | 91 | 40 | 47 |
| | 49 | 103 | 37 | 35 |
| CF508-48T | 186 | 32 | 48 | 34 |
| | 180 | 38 | 63 | 41 |
| | 183 | 36 | 43 | 33 |
| | 179 | 33 | 55 | 45 |
| Avg. ctrl. | 53 | 36 | 46 | 38 |
| S.D. Ctrl. | 4.0 | 2.5 | 8.3 | 5.3 |
| Avg. net chg.^{b} | 129 | 57 | 74 | 55 |

Positive reactions are in boldface type.
^{a}FP measurements for FAM were made with excitation at 485 nm and monitored at 530 nm.
^{b}Net change over average of control.

In all cases, only the nucleoside complementary to the polymorphic base was incorporated and showed significant FP change, with net gains of FP of at least 50 mP, which is nine times standard deviation of the controls.

FP-EcoN I Assay with Different Terminators Labeled with Different Dyes

To identify different dyes suitable for multicolor detection in the same reaction, a number of different dyes were studied for their FP properties in the FP-EcoN I assay. With all the combinations of dye-terminators tested, the optimal set of terminators, chosen for minimal standard deviations in the control samples and large net changes in the positive samples, were found to be BODIPY-fluorscein-ddA (BFL-ddA), N,N,N′, N′-tetamethyl-6-carboxyrhodamine (TMR-ddC), b-carboxy-x-rhodamine-ddG (ROX-ddG), and BODIPY-Texas Red-ddU (BTR-ddU) (see Table 3). In all of these cases, the net increase in FP exceeded 10 times standard deviation of the mean of the control samples. In addition, BFL-ddC, BFL-ddT, ROX-ddA, BTR-ddC, TMR-ddU, and all FAM terminators also worked.

TABLE 3
FP-EcoN I Assay with Synthetic Templates
Using Different Dye Terminators

| | BFL-ddA | TMR-ddC | ROX-ddG | BTR-ddU |
|---|---|---|---|---|
| Templates | (mP)^{a} | (mP)^{a} | (mP)^{a} | (mP)^{a} |
| CFSO8-48A | 38 | 43 | 77 | 174 |
| | 37 | 53 | 73 | 175 |
| | 31 | 36 | 78 | 174 |
| | 35 | 49 | 82 | 170 |
| CF508-48C | 20 | 50 | 214 | 32 |
| | 19 | 37 | 209 | 27 |
| | 20 | 56 | 215 | 25 |
| | 14 | 38 | 207 | 26 |
| CF508-48G | 23 | 247 | 84 | 23 |
| | 24 | 266 | 80 | 30 |
| | 22 | 253 | 75 | 23 |
| | 15 | 262 | 74 | 21 |
| CF508-48T | 113 | 52 | 81 | 32 |
| | 106 | 41 | 68 | 39 |
| | 108 | 59 | 81 | 30 |
| | 103 | 32 | 76 | 28 |
| Avg. ctrl. | 25 | 46 | 86 | 28 |
| S.D. Ctrl. | 8.2 | 8.8 | 4.6 | 5.0 |
| Avg. net chg.^{b} | 83 | 211 | 134 | 145 |

Positive reactions are in boldface type.
^{a}FP measurements for BFL were made with excitation at 480 nm and emission at 520 nm; for TMR the excitation was 535 nm and emission was 590 nm; for BTR the excitation was 591 nm and emission was 635 nm; for ROX the excitation was 580 nm and emission was 620 nm.
^{b}Net change over average of control.

Dual Color Fp-EcoN I Assay for Amplified Genomic DNA

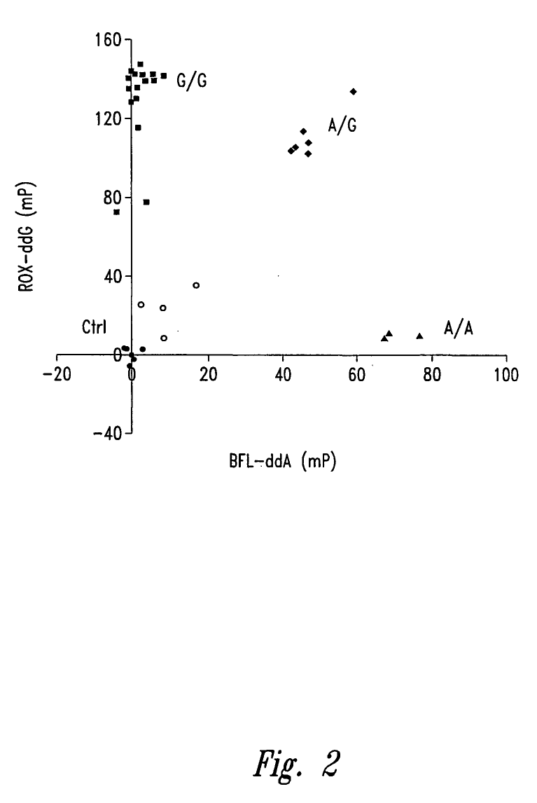

Marker D18S8 and the C282Y mutation in the human hereditary hemochromatosis (HFE) gene implicated in hemochromatosis were used in FP-TDI assays designed to test for both alleles in the same reaction. For marker D18S8, genomic DNA samples from 34 individuals were amplified and then cut with EcoN I and the 3′-ends filled in, in the presence of BFL-ddA and ROX-ddG. The FP values of the reaction mixtures were read at the BFL and ROX emission wavelengths, respectively, and the results are plotted and shown in FIG. 2 as changes in fluorescence polarization. The results are plotted in mP units above the average polarization of the negative controls. A change of 40 mP for a dye-terminator is scored as positive. DNA samples from 34 individuals and 6 water blanks were used. (▪) Samples positive for the G allele but negative for the A allele (homozygous G); (▴) samples positive for the A allele but negative for the G allele (homozygous A); (♦) samples positive for both alleles (heterozygotes); (●) negative controls; (◯) samples with failed PCR amplification.

The FP values cluster into four groups. In the upper left corner of the plot, the samples have high FP for ROX-ddG but low FP for BFL-ddA, signifying that they are of homozygous G genotype (▪). The heterozygous A/G samples (♦) exhibit high FP values in both BFL-ddA and ROX-ddG and occupy the right upper corner of the plot. The homozygous A/A samples (▴) are found in the lower right corner, with low ROX-ddG but high BFL-ddA FP values. The negative controls (●) and samples with failed PCR reactions (◯) occupy the area near the origin with low FP values for both dyes. The positive samples in both the BFL-ddA and the ROX reactions gave FP values that were more than 40 mP and 100 mP above average of controls, respectively. These values were more than 20 times standard deviation of the controls and the genotypes of the samples were easily assigned. Of 34 test samples, 4 gave inconclusive results because of PCR failure, which would prevent analysis by any method, including those based on gel electrophoresis.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for identifying a nucleotide at a defined position in a single-stranded target nucleic acid, comprising
(a) forming a mixture of a first oligonucleotide primer (ODNP), a second ODNP, and the target nucleic acid, wherein
the first ODNP comprises a nucleotide sequence that is complementary to a nucleotide sequence of the target nucleic acid at a location 3′ to the defined position,
the second ODNP comprises a nucleotide sequence that is complementary to a nucleotide sequence of the complement of the target nucleic acid at a location 3′ to the complementary nucleotide of the nucleotide at the defined position, and
the first and second ODNPs further comprise a first constant recognition sequence (CRS) of a first strand and a second CRS of a second strand of an interrupted restriction endonuclease recognition sequence (IRERS), respectively, but not a complete IRERS, the complete IRERS being a double-stranded nucleic acid having the first and the second strands and comprising the first and the second constant recognition sequences (CRS) linked by a variable recognition sequence (VRS);
(b) extending the first and second ODNPs to form a fragment having the complete IRERS wherein the nucleotide to be identified is within the VRS;
(c) cleaving the fragment with a restriction endonuclease that recognizes the complete IRERS, and thereby producing a 5′-overhang consisting of either the nucleotide to be identified or the complement thereof;
(d) filing in the 3′ recessed terminus corresponding to the 5′ overhang with a fluorescence labeled nucleoside triphosphate; and
(e) detecting the incorporated fluorescence labeled nucleotide with fluorescence polarization.

2. The method of claim 1 wherein the defined position is polymorphic.

3. The method of claim 1 wherein a mutation at the defined position is associated with a disease.

4. The method of claim 3 wherein the disease is a human genetic disease.

5. The method of claim 1 wherein a mutation at the defined position is associated with drug resistance of a pathogenic microorganism.

6. The method of claim 1 wherein the single-stranded target nucleic acid is one strand of a denatured double-stranded nucleic acid.

7. The method of claim 6 wherein the double-stranded nucleic acid is genomic nucleic acid.

8. The method of claim 6 wherein the double-stranded nucleic acid is cDNA.

9. The method of claim 1 wherein the single-stranded target nucleic acid is derived from the genome of a pathogenic virus.

10. The method of claim 1 wherein the single-stranded target nucleic acid is derived from the genome or episome of a pathogenic bacterium.

11. The method of claim 1 wherein the target nucleic acid is synthetic nucleic acid.

12. The method of claim 1 wherein the nucleotide sequence of the first ODNP complementary to the target nucleic acid is at least 12 nucleotides in length.

13. The method of claim 1 wherein the nucleotide sequence of the second ODNP complementary to the complement of the target nucleic acid is at least 12 nucleotides in length.

14. The method of claim 1 wherein the first ODNP is 15-85 nucleotides in length.

15. The method of claim 1 wherein the second ODNP is 15-85 nucleotides in length.

16. The method of claim 1 wherein the first ODNP further comprises one or more nucleotides complementary to the target nucleic acid at the 3′ terminus of the first CRS.

17. The method of claim 1 wherein the second ODNP further comprises one or more nucleotides complementary to the target nucleic acid at the 3′ terminus of the second CRS.

18. The method of claim 1 wherein step (b) is carried out by performing a polymerase chain reaction.

19. The method of claim 1 wherein step (d) is carried out with a RNA polymerase.

20. The method of claim 1 wherein step (d) is carried out with a DNA polymerase.

21. The method of claim 20 wherein the fluorescence labeled nucleoside triphosphate is deoxynucleoside triphosphate.

22. The method of claim 20 wherein the fluorescence labeled nucleoside triphosphate is dideoxynucleoside triphosphate.

23. The method of claim 22 wherein the fluorescence labeled dideoxynucleoside triphosphate is any one selected from the group consisting of FAM-ddA, FAM-ddU, FAM-ddC, FAM-ddG, BODIPY-fluorescein-ddA (BFL-ddA), tetramethylrhodamine-ddc (TMR-ddC), b-caboxy-x-prhodamine-ddG (ROX-ddG), and BODIPY-Texas Red-ddU (BTR-ddU).

24. The method of claim 1 wherein the 5′ terminus of either said first ODNP or said second ODNP is linked to a biotin molecule.

25. The method of claim 1 further comprising separating products of step (d) before the detection of step (e).

26. The method of claim 1 wherein the IRERS is recognizable by a restriction endonuclease selected from the group consisting of PflF I, EcoN I, Fnu4H I, ScrF I, and Tth111 I.

27. An oligonucleotide primer, comprising
(a) a first CRS of a first strand of an IRERS, but not the first strand of a complete IRERS, wherein the complete IRERS is a double-stranded oligonucleotide having the first strand and a second strand, the complete IRERS comprises the first CRS and a second CRS linked by a VRS having a number n of variable nucleotides, and the digestion at the IRERS produces a 5′ overhang consisting of a single nucleotide; and
(b) at a location 5′ to the 5′ terminus of the first CRS, an oligonucleotide sequence complementary to a nucleotide sequence of a single-stranded target nucleic acid at a location 3′ to a defined position, wherein when the oligonucleotide sequence anneals to the target nucleic acid, the distance between the nucleotide in the target corresponding to the 3′ terminal nucleotide of the primer and the defined position is within the range 0 to (n-1)/2.

28. The primer of claim 27 wherein oligonucleotide sequence (b) is at least 12 nucleotides in length.

29. The primer of claim 27 wherein the primer is 15-85 nucleotides in length.

30. The primer of claim 27 wherein the primer further comprises one or more nucleotides complementary to the target nucleic acid at the 3′ terminus of the first CRS.

31. The oligonucleotide primer of claim 27 wherein the IRERS is recognizable by EcoN I.

32. The primer of claim 27 wherein the defined position in the target nucleic acid is polymorphic.

33. The primer of claim 27 wherein a mutation at the defined position in the target nucleic acid is associated with a disease.

34. The primer of claim 27 wherein the target nucleic acid is one strand of a denatured double-stranded nucleic acid.

35. The primer of claim 34 wherein the double-stranded nucleic acid is either genomic nucleic acid or cDNA.

36. The primer of claim 27 wherein the 5′ terminus of the primer is linked to a biotin molecule.

37. An oligonucleotide primer pair for producing a portion of a single-stranded target nucleic acid containing a nucleotide to be identified at a defined position, comprising first and second ODNPs wherein
the first ODNP comprises a nucleotide sequence complementary to a nucleotide sequence of the target nucleic acid at a location 3′ to the defined position;
the second ODNP comprises a nucleotide sequence complementary to a nucleotide sequence of the complement of the target nucleic acid at a location 3′ to the complementary nucleotide of the nucleotide to be identified;
the first and second ODNPs further comprise a first constant recognition sequence (CRS) of a first strand and a second CRS of a second strand of an interrupted restriction endonuclease recognition sequence (IRERS), respectively, but not a complete IRERS, the complete IRERS being a double-stranded nucleic acid having the first and the second strands and comprising the first and the second constant recognition sequences (CRS) linked by a variable recognition sequence (VRS); and
a fragment resulting from an amplification of the first and second ODNPs comprises a complete IRERS, and digestion at the IRERS produces a 5′ overhang consisting of either the nucleotide to be identified or the complement thereof.

38. The primer pair of claim 37 wherein the nucleotide sequence complementary to the target nucleic acid of the first ODNP is at least 12 nucleotides in length.

39. The primer pair of claim 37 wherein the nucleotide sequence complementary to the complement of the target nucleic acid of the second ODNP is at least 12 nucleotides in length.

40. The primer pair of claim 37 wherein either the first ODNP or the second ODNP is 15-85 nucleotides in length.

41. The primer pair of claim 37 wherein the first ODNP further comprises one or more nucleotides complementary to the target nucleic acid at the 3′ terminus of the first CRS.

42. The primer pair of claim 37 wherein the second ODNP further comprises one or more nucleotides complementary to the target nucleic acid at the 3′ terminus of the second CRS.

43. The primer pair of claim 37 wherein the IRERS is recognizable by EcoN I.

44. The primer pair of claim 37 wherein the defined position in the target nucleic acid is polymorphic.

45. The primer pair of claim 37 wherein a mutation at the defined position in the target nucleic acid is associated with a disease.

46. The primer pair of claim 37 wherein the target nucleic acid is one strand of a denatured double-stranded nucleic acid.

47. The primer pair of claim 37 wherein the double-stranded nucleic acid is either genomic nucleic acid or cDNA.

48. The primer pair of claim 37 wherein the 5′ terminus of either said first ODNP or said second ODNP is linked to a biotin molecule.

49. A composition comprising the primer according to any one of claims 27-36 and the target nucleic acid.

50. A kit comprising the primer pair according to any one of claims 37-48.

51. The kit of claim 50 further comprises a restriction endonuclease that recognizes the IRERS.

52. The kit of claim 50 further comprises instruction of use thereof.

53. A set of two ODNP pairs, comprising first and second ODNP pairs each comprising first and second ODNPs wherein:
(a) the first ODNP in the first ODNP pair comprises
an oligonucleotide sequence complementary to a nucleotide sequence of a single-stranded target nucleic acid at a location 3′ to a defined position in the target nucleic acid, and
a first CRS of a first strand of an IRERS, but not the first strand of a complete IRERS, the complete IRERS being a double-stranded nucleic acid having first and second strands and comprising the first CRS and a second CRS linked by a VRS;
(b) the second ODNP in the first ODNP pair comprises
an oligonucleotide sequence complementary to a nucleotide sequence of the target nucleic acid at a location 5′ to the defined position, and
a second CRS of the first strand of the IRERS, but not the first strand of the complete IRERS;
(c) the first ODNP in the second ODNP pair comprises
an oligonucleotide sequence complementary to a nucleotide sequence of the complement of the target nucleic acid at a location 5′ to the position in the complement corresponding to the defined position in the target nucleic acid, and
a first CRS of the second strand of the IRERS, but not the second strand of the complete IRERS; and
(d) the second ODNP in the second ONDP pair comprises
an oligonucleotide sequence complementary to a nucleotide sequence of the complement of the target nucleic acid at a location 3′ to the position in the complement corresponding to the defined position in the target nucleic acid, and
a second CRS of the second strand of the IRERS, but not the second strand of the complete IRERS; and
(e) a fragment resulting from an extension and ligation of the first and second ODNPs in each ODNP pair comprises the complete IRERS, wherein digestion at the IRERS produces a 5′ overhang consisting of either the nucleotide at the defined position or the complement thereof.

54. A method comprising:
(a) providing a double-stranded nucleic acid molecule comprising an interrupted restriction endonuclease recognition sequence (IRERS), wherein the IRERS comprises a first constant recognition sequence (CRS) and a second CRS linked by a variable recognition sequence (VRS);
(b) cleaving the nucleic acid molecule with a restriction endonuclease that recognizes the IRERS, and thereby producing a fragment with a 5′ overhang consisting of either a nucleotide to be identified at a defined position in the nucleic acid molecule or the complementary nucleotide thereof;
(c) filing in the 3′ recessed terminus corresponding to the 5′ overhang with a fluorescence labeled nucleoside triphosphate; and
(d) detecting the incorporated fluorescence labeled nucleotide with fluorescence polarization.

55. The method of claim 54, wherein step (a) comprises
(i) forming a mixture of the primer pair set of claim 53 and the target nucleic acid;
(ii) extending the first and second ODNPs of the first and second ODNP pairs;
(iii) ligating the extended products of step (ii); and
(iv) amplifying the fragments of step (iii).

56. The method of claim 54, wherein step (a) comprises
(i) forming a mixture of the primer pair of claim 46 and the target nucleic acid; and
(ii) extending the first and the second ODNPs.

57. The method of claim 54, wherein step (a) comprises
(i) forming a mixture of a first ODNP, a second ODNP and a single-stranded target, wherein
the first ODNP comprises
an oligonucleotide sequence complementary to a nucleotide sequence of the target nucleic acid at a location 3′ to a defined position in the target nucleic acid, and
a first CRS of a first strand of an IRERS, but not the first strand of a complete IRERS, the complete IRERS being a double-stranded nucleic acid having first and second strands and comprising the first CRS and a second CRS linked by a VRS,
the second ODNP comprises
an oligonucleotide sequence complementary to a nucleotide sequence of the target nucleic acid at a location 5′ to the defined position, and
a second CRS of the first strand of the IRERS, but not the first strand of the complete IRERS;
(ii) extending the first and second ODNPs;
(iii) ligating the extended products of step (ii);
(iv) denaturing the ligation product of step (iii); and
(v) annealing the denatured ligation product of step (iv) that contains said first and second ODNPs with an oligonucleotide that has a universe nucleotide at the position corresponding to the defined position in the double stranded nucleic acid molecule, wherein the resulting double-stranded nucleic acid molecule comprises an complete IRERS.

58. The method of claim 54 wherein the defined position is polymorphic.

59. The method of claim 54 wherein a mutation at the defined position is associated with a disease.

60. The method of claim 59 wherein the disease is a human genetic disease.

61. The method of claim 54 wherein a mutation at the defined position is associated with drug resistance of a pathogenic microorganism.

62. The method of claim 54 wherein step (c) is carried out with a RNA polymerase.

63. The method of claim 54 wherein step (c) is carried out with a DNA polymerase.

64. The method of claim 63 wherein the fluorescence labeled nucleoside triphosphate is deoxynucleoside triphosphate.

65. The method of claim 63 wherein the fluorescence labeled nucleoside triphosphate is dideoxynucleoside triphosphate.

66. The method of claim 65 wherein the fluorescence labeled dideoxynucleoside triphosphate is any one selected from the group consisting of FAM-ddA, FAM-ddU, FAM-ddC, FAM-ddG, BODIPY-fluorescein-ddA (BFL-ddA), tetramethylrhodamine-ddC (TMR-ddC), b-caboxy-x-prhodamine-ddG (ROX-ddG), and BODIPY-Texas Red-ddU (BTR-ddU).

67. The method of claim 54 further comprising separating products of step (c) before the detection of step (d).

68. The method of claim 54 wherein the IRERS is recognizable by a restriction endonuclease selected from the group consisting of PflF I, EcoN I, Fnu4H I, ScrF I, and Tth111 I.

69. A method comprising the steps:
(a) combining a first ODNP, a second ODNP, and a single stranded target nucleic acid under primer extension conditions, wherein
the first ODNP comprises
an oligonucleotide sequence complementary to a nucleotide sequence of the target nucleic acid at a location 3′ to a defined position in the a target nucleic acid, and
a first CRS of a first strand of an IRERS, but not the first strand of a complete IRERS, the complete IRERS being a double-stranded nucleic acid having first and second strands and comprising the first CRS and a second CRS linked by a VRS,
the second ODNP comprises
an oligonucleotide sequence complementary to a nucleotide sequence of the target nucleic acid at a location 5′ to the defined position, and
a second CRS of the first strand of the RERS, but not the first strand of the complete IRERS;
(b) performing at least three rounds of primer extension to provide a primer extension product;
(c) cleaving the primer extension product with a restriction endonuclease that recognizes an IRERS and thereby producing a 5′ overhang consisting of either the nucleotide at the defined position or the complement thereof;
(d) filling in the 3′ recessed terminus corresponding to the 5′ overhang with a fluorescence labeled nucleoside triphosphate; and
(e) detecting the incorporated fluorescence labeled nucleotide with fluorescence polarization.

70. The method of claim 69 wherein the defined position is associated with a disease.

71. The method of claim 69 wherein the single-stranded nucleic acid is one strand of a denatured double-stranded nucleic acid.

72. The method of claim 69 wherein the double-stranded nucleic acid is either genomic DNA or cDNA.

73. The method of claim 69 wherein the nucleotide sequence of the first ODNP complementary to the target nucleic acid is at least 12 nucleotides in length.

74. The method of claim 69 wherein the nucleotide sequence of the second ODNP complementary to the target nucleic acid is at least 12 nucleotides in length.

75. The method of claim 69 wherein the first ODNP further comprises one or more nucleotides complementary to the target nucleic acid at the 3′ terminus of the first CRS.

76. The method of claim 69 wherein the second ODNP further comprises one or more nucleotides complementary to the target nucleic acid at the 3′ terminus of the second CRS.

77. The method of claim 69 wherein step (b) is carried out by performing a polymerase chain reaction.

78. The method of claim 69 wherein step (d) is carried out with a RNA polymerase.

79. The method of claim 69 wherein step (d) is carried out with a DNA polymerase.

80. The method of claim 79 wherein the fluorescence labeled nucleoside triphosphate is deoxynucleoside triphosphate.

81. The method of claim 79 wherein the fluorescence labeled nucleoside triphosphate is dideoxynucleoside triphosphate.

82. The method of claim 69 wherein the fluorescence labeled dideoxynucleoside triphosphate is any one selected from the group consisting of FAM-ddA, FAM-ddU, FAM-ddC, FAM-ddG, BODIPY-fluorescein-ddA (BFL-ddA), tetramethylrhodamine-ddC (TMR-ddC), b-caboxy-x-prhodamine-ddG (ROX-ddG), and BODIPY-Texas Red-ddU (BTR-ddU).

83. The method of claim 69 wherein the 5′ terminus of either said first ODNP or said second ONP is linked to a biotin molecule.

84. The method of claim 69 further comprising separating products of step (d) before the detection of step (e).

85. The method of claim 69 wherein the IRERS is recognizable by a restriction endonuclease selected from the group consisting of PflF I, EcoN I, Fnu4H I, ScrF I, and Tth111 I.


SEQUENCE LISTING (10 sequences)

SEQ ID NO: 1 - DNA, 11 bp, Unknown Organism
  DNA motif cleaved by EcoN I restriction enzyme
cctnnnnnagg

SEQ ID NO: 2 - DNA, 11 bp, Unknown
  DNA motif cleaved by EcoN I restriction enzyme
cctnnnnnagg

SEQ ID NO: 3 - DNA, 48 bp, Artificial Sequence
  Synthetic template used in FP studies
atattcatcataggaaacctcaaagaggatattttctttaatggtgcc

SEQ ID NO: 4 - DNA, 48 bp, Artificial Sequence
  Synthetic template used in FP studies
atattcatcataggaaacctcacagaggatattttctttaatggtgcc

SEQ ID NO: 5 - DNA, 48 bp, Artificial Sequence
  Synthetic template used in FP studies
atattcatcataggaaacctcagagaggatattttctttaatggtgcc

SEQ ID NO: 6 - DNA, 48 bp, Artificial Sequence
  Synthetic template used in FP studies
atattcatcataggaaacctcatagaggatattttctttaatggtgcc

SEQ ID NO: 7 - DNA, 20 bp, Artificial Sequence
  PCR primer used in FP studies
tggcaagggtaaacagatcc

SEQ ID NO: 8 - DNA, 20 bp, Artificial Sequence
  PCR primer used in FP studies
ctcaggcactcctctcaacc

SEQ ID NO: 9 - DNA, 21 bp, Artificial Sequence
  PCR primer used in FP studies
ttgcaccatgctgaagattgt

SEQ ID NO: 10 - DNA, 20 bp, Artificial Sequence
  PCR primer used in FP studies
accctccccctgatgactta